(19) United States
(12) Patent Application Publication
(10) Pub. No.: US 2021/0283563 A1
(43) Pub. Date: Sep. 16, 2021

(54) EMULSION COMPRISING ANTIOXIDANT PARTICLES

(71) Applicants: Naturex S.A., Avignon (FR); Wageningen University, Wageningen (NL)

(72) Inventors: Albertine Johanneke SCHRÖDER, Wageningen (NL); Joris Henricus Bernadus SPRAKEL, Wageningen (NL); Catharina Gerarda Petronella Henrica SCHROEN, Wageningen (NL); Claire BERTON-CARABIN, Wageningen (NL); Mickaël LAGUERRE, Le Pontet (FR); Simona BIRTIC, Cavaillon (FR)

(21) Appl. No.: 17/256,875

(22) PCT Filed: Jul. 2, 2019
(86) PCT No.: PCT/EP2019/067780

§ 371 Date: Dec. 29, 2020

(30) Foreign Application Priority Data
Jul. 2, 2018 (GB) 1810850.6
Apr. 17, 2019 (GB) 1905441.0

(51) Int. Cl. B01F 17/00; C11B 5/00; A61K 9/107; A61K 47/44; A61K 8/06; A61K 8/92; A61Q 19/00; A23D 7/005; A23D 7/06; A23D 7/04; A23L 33/115; A23K 20/158; A23L 2/52

(52) U.S. Cl. CPC B01F 17/0078; A23V 2002/00; C11B 5/0085; C11B 5/0092; A61K 9/107; A61K 47/44; A61K 8/062; A61K 8/922; A61Q 19/00; A61K 9/1075; A23D 7/0053; A23D 7/06; A23D 7/04; A23L 33/115; A23K 20/158; A23L 2/52; A61K 2800/522; A61K 2800/10; A61K 2800/412; A61K 2800/21; B01F 17/0021

(57) ABSTRACT

The present invention relates to compositions comprising particles prepared from one or more biological materials and/or animal lipids and/or plant lipids that are capable of locating to an interface when combined with two or more immiscible liquids. Emulsions comprising the compositions comprising particles, wherein the emulsion has an internal phase dispersed in a continuous external phase and the particles are located at the interface of the external and the internal phase, methods of preparing such compositions and emulsions, the use of such compositions and emulsions and products containing the compositions and emulsions are also described.

The present invention relates to compositions comprising particles prepared from one or more biological materials, which particles are capable of locating to an interface when combined with two or more immiscible liquids. Emulsions comprising the compositions comprising particles, wherein the emulsion comprises an internal phase dispersed in a continuous external phase and the particles are located at the interface of the external and the internal phase, methods of preparing such compositions and emulsions, the use of such compositions and emulsions and products containing the compositions and emulsions are also described.

Emulsions, such as oil-in-water emulsions, are systems that are made when two immiscible liquids are mixed together creating an internal phase dispersed in a continuous external phase. For example, oil and water in an oil-in-water emulsion. The area between internal and external phases is referred to as the interface.

Emulsions, such as oil-in-water emulsions, are thermodynamically unstable systems in which the internal phase must be physically stabilized to avoid phase separation.

Surfactant molecules (referred to hereinafter as ‘conventional emulsifiers’) can stabilize emulsions by adsorbing at the interface during a homogenization step because of their high affinity for the interface. The surfactant adsorption decreases the interfacial tension between the internal and external phases, thereby reducing the total free energy of the system.

As used herein, the term “stabilizing” when referring to emulsions means preventing the separation of the two immiscible liquids present in the emulsion.

Emulsifiers are typically divided into two groups:

(i) small surfactants (<2000 g/mol) which contain both a hydrophilic group (such as a positively or negatively charged moiety, a sugar or a sugar-derived moiety, or a polyoxyethylene chain) and a hydrophobic group (such as an alkyl chain), and
(ii) proteins which are heteropolymers made of aminoacids.

‘Pickering particles’ may also be used to stabilize emulsions. It is believed that the particles anchor in the interface and provide a mechanical (steric) barrier protecting the lipid droplets against coalescence.

If the emulsion comprises a lipid phase, for example in an oil-in-water emulsion, and the lipid phase is susceptible to oxidation, i.e. contains unsaturated lipids (lipids containing at least one carbon-carbon double bond), lipid oxidation can occur decreasing both nutritional and sensory quality of the product (Laguerre, Bily, Roller, Birtic. Mass transport phenomena in lipid oxidation and antioxidation. Annu. Rev. Food Sci. Technol. 2017, 8, 391-411).

The listing or discussion of an apparently prior-published document in this specification should not necessarily be taken as an acknowledgement that the document is part of the state of the art or common general knowledge.

The present invention provides a composition comprising particles prepared from one or more biological materials, which particles are capable of locating to, or are located at, an interface when combined with two or more immiscible liquids.

By the term “located at an interface”, we mean that a significant proportion of the particles (e.g. at least 50% by weight of the particles, such as at least 60%, 70%, 80%, 90%, 95% or 99%) are located at the interface between two immiscible liquids. Particles that have this property include those disclosed herein. Such particles are typically substantially insoluble in the two or more immiscible liquids (e.g. less than 25% of the particle dissolves in any of the two or more immiscible liquids, such as less than 10% or less than 5% or less than 1%).

The particles may comprise one or more amphiphilic compounds, e.g. of the sort described elsewhere herein.

This composition comprising particles is hereinafter referred to as the composition of the invention.

The composition of the invention may consist of or consist essentially of particles prepared from one or more biological materials that are capable of locating to, or are located at, an interface when homogenised with two or more immiscible liquids.

The composition may be dry, i.e. in the form of a powder, or may be a liquid, e.g. in the form of a solid suspension in a liquid or a colloidal dispersion of a solid in a liquid.

The composition may comprise the particles prepared from one or more biological materials in an amount of from about 0.1 to about 100% by weight of the composition, such as from about 1 to about 80% or from about 10 to about 60%.

For example, where the composition is in dry form, the particles may be present in an amount from about 1 to about 100% by weight of the composition, such as from about 5% to about 95% or from about 10% to about 90%.

Where the composition is in the form of a liquid, the particles may be suspended (suspension) or dispersed (colloidal dispersion) in the liquid. In the composition, where the composition is in the form of a liquid, the particles may be present in an amount from about 0.1 to about 60% by weight of the composition, such as from about 1% to about 40%.

The particles may be biological material such as lipids (for example high melting point lipids, where the particles may be obtained via a process as defined herein) or biological material comprising less than 50% lipid, such as less than 25% lipid or less than 10% lipid by weight of the biological material, in the form of powder or particles that are capable of locating to an interface when homogenised with two or more immiscible liquids.

The lipid may comprise or encapsulate a natural material (e.g. plant extract) or compound (e.g. compounds extracted from a natural source) that are not capable of forming particles by themselves when homogenised with two or more immiscible liquids due to their solubility in at least one of the two or more immiscible liquids. For example, the lipid may encapsulate a natural material or compound where more than 50% of the natural material or compound is soluble in the two or more immiscible liquids, such as more than 75% or more than 90% by weight of the material or compound.

Typically, the natural material or compound will have a desirable property. For example, the natural material or compound may comprise anti-oxidant activity.

As used herein, the term “high melting point” means that the lipid or a significant part of a lipid mixture, such as more than 25% by weight of the mixture, or more than 50% by weight of the mixture is solid at room temperature.

As used herein, the term “room temperature” means a temperature suitable for human occupancy, typically from about 15° C. to about 35° C. or from about 20° C. to about 25° C.

In the particles prepared from biological material, the biological material may be modified such that it is distinct from the form that it is found in nature. For example, the biological material may be modified as to remove water and/or other compounds present in the biological material in order to concentrate the compounds remaining in the biological material. The modification may typically result in the biological material having activity that is not present in the unmodified material or is enhanced when compared to the unmodified material. For example, the biological material may be extract using a specific solvent which results in only certain compounds being solubilized and extracted from the biological material.

The biological material may be obtained from photosynthetic organisms. For example, the biological material may be obtained from plants or may be obtained from algae, such as blue-green algae. The biological material obtained from photosynthetic organisms may be in the form of a plant or algae extract, raw plant or algae material (dried or undried), powder, by-product of extract or extraction cake.

As used herein, the term “plant extract” includes any plant material that has been extracted from plants, such as from the roots, aerial parts, leaves, flowers, stems, barks, fruits, branches or seeds or their tissues using a solvent, such as water, organic solvents and mixtures thereof.

For example, the particles prepared from biological material may be obtained from (e.g. are an extract of) one or more photosynthetic organisms including Zingiberaceae (e.g. turmeric), Lamiaceae (e.g. rosemary), Brassicaceae (e.g. radish), Cyanobacteria (e.g. spirulina), Camellia (e.g. green tea), Bromeliaceae (e.g. pineapple) and Amaranthaceae (e.g. spinach).

As used herein, the term “raw material” means material that has been directly obtained without being chemically modified or has only be subjected to a drying step to remove from about 10 to about 100% of the water present, such as from about 20 to about 90% or from about 30 to about 80% by weight of the raw material. The raw material may be physically modified, for example, the raw material may be ground or micronized.

The biological material may also be obtained from animal (including fish) lipids and/or plant lipids.

As used herein, the term “animal lipids” include any lipids and mixtures that are derived from an animal source. For example, butter, milk fats and beeswax.

As used herein, the term “plant lipids” include any lipids or mixtures that are derived from a plant source. For example, plant lipids may include those selected from the group consisting of palm oil, palm kernel oil, coconut oil, cuphea oil, cocoa butter, Pentadesma butter, shea butter and plant waxes.

Plant waxes used to prepare the colloidal particles described in this invention maybe (i) hydrocarbons (alkanes), (ii) long-chain fatty acid linked through an ester bond to a long-chain alcohol, (iii) very long chain alcohols, or (iv) very long chain fatty acids. For example, plant waxes used to prepare the colloidal particles described in this invention may be selected from the group consisting of jojoba wax, carnauba wax, candelilla wax, microscrystalline wax, rice bran wax, soya wax, sugar cane wax, shellac wax, grain sorghum wax, Bayberry wax, and Eucalyptus leaf wax.

Fats with a high melting point and which are obtained by fractionation or hydrogenation of vegetable oils can also be used to prepare the colloidal particles described in this invention. They may include the product of the hydrogenation of palm oil, coconut oil, palm kernel oil, corn oil, soybean oil, sunflower oil, rapeseed oil, and mixtures thereof. They may also include fractionated oil such as palm oil (palm stearin), or palm kernel oil (palm kernel stearin), and mixtures thereof. Furthermore, purified fat such as tripalmitin can also be used.

The particles may be prepared from animal and/or plant lipids having a high melting point, such as those coming from animal fats such as milk fat or those naturally found in vegetable oils. Fats with a high melting point and which are obtained by fractionation or hydrogenation of vegetable oils can also be used to prepare the colloidal particles described in this invention.

Typically, the particles prepared from biological material may be in the form of a solid powder/particles.

The particles prepared from biological material may be colloidal particles and/or Pickering particles.

As used herein the term ‘Pickering particles’ means any solid or semi solid particles which are not soluble in the external or the internal phase of an emulsion and are predominately (i.e. at least 50% of the particles, such as at least 75% of the particles or at least 90% of the particles) located at the interface between the external and the internal phase, wherein at least one of the internal or external phase comprises an oxidisable material and the particles are prepared from a biological material as defined herein.

The particles formed from biological material may comprise a mixture of compounds depending on the biological source.

For example, where the biological material used to prepare the particles originates from plant or algal material, the particles may contain plant or algal derived chemical constituents selected from the groups consisting of: lignin, cellulose, hemicellulose, alkaloids, glycosides, organic acids, resins (including resin acids, resin alcohols and hydrocarbon resins), volatile oils, sugars (including starches, inulin, gums and phlegmatic, etc.), amino acids, proteins and enzymes, phenolic compounds, tannins, plant pigments (including chlorophyll, carotenoids, flavonoids, beet red bases and quinones, etc.), oils and waxes, inorganic ingredients (trace elements) and mixtures thereof.

The particles present in the composition may be micron or submicron size. For example, the particles may predominantly (such as more than 70%, more than 80% or more than 90% of the particles in the composition) have a diameter from about 0.1 μm to about 100 μm, such as from about 0.2 μm to about 50 μm or from about 0.5 μm to about 30 μm as measured using droplet size distribution measurement such as DLS (dynamic light scattering) and imaging microscopy such as TEM (transmission electronic microscopy) or light microscopy. It is also to be understood that the composition may comprise small amounts (such as less than 30%, less than 20%, less than 10% of the particles in the composition) of particles below the lower diameter and above the higher diameter.

Typically, the particles present in the composition are not nano-scale, where nano-scale is intended to mean particles having a diameter of from about 1 nm to about 99 nm. For example, 50% or less of the particles have a diameter from about 1 nm to about 99 nm, such as 40% or less, 30% or less, 20% or less, 10% or less or 1% or less.

Each particle present in the emulsion may contain a liquid lipid phase within each individual particle. For example, each particle (such as particles obtained from animal lipids and/or plant lipids, typically high melting point lipids) may contain/encapsulate a fraction comprising tricaprylin, vegetable oils liquid at room temperature (such as sunflower, canola, soybean oil), and/or medium chain triglycerides. The liquid lipid phase may contain at least one anti-oxidant.

In the composition, the particles may be prepared from biological material selected from the group consisting of blue-green algae (spirulina), the Rutaceae family (including Citrus such as orange, lime or lemon), the Malvaceae family (including cocoa and marshmallow), the Rubiaceae family (including coffee), the Amaranthaceae family (including beetroot and spinach), the Poaceae family (including bamboo and oat), the Zingiberaceae family (including curcuma), the Ginkgoaceae (including ginkgo), the Araliaceae family (including ginseng), the Theaceae (including matcha tea), the Asteraceae family (including milk thistle), the Oleaceae family (including olive tree), the Moringaceae family (including moringa), the Bromeliaceae family (including pineapple), the Brassicaceae family (including red radish), the Rosaceae family (including rosehip), the Sapindaceae family (including guarana), and the Lamiacea family (including rosemary, sage, thyme, basil, and oregano), and mixtures thereof and/or animal lipids and/or plant lipids selected from the group consisting of butter, milk fat, beeswax, palm oil, palm kernel oil, coconut oil, cuphea oil, cocoa butter, Pentadesma butter, shea butter and plant waxes as defined above.

Fats with a high melting point and which are obtained by fractionation or hydrogenation of vegetable oils can also be used to prepare the particles described in this invention. They may include the product of the hydrogenation of palm oil, coconut oil, palm kernel oil, corn oil, soybean oil, sunflower oil, rapeseed oil, and mixtures thereof. They may also include fractionated oil such as palm oil (palm stearin), or palm kernel oil (palm kernel stearin), and mixtures thereof. Furthermore, purified fat such as tripalmitin can also be used.

In the composition, the particles may comprise an antioxidant.

As used herein, the term ‘antioxidant’ means any molecule, or group of molecules, or extract obtained from a biological material, which is able, when present in a formulation (such as an emulsion), to prevent or delay the oxidation of an oxidizable substrate.

In the composition, the antioxidant may be endogenous to the biological material or may be added to the biological material, i.e. present in the biological material used to make the particle or may have been added to the particle. For example, particles obtained from biological materials may inherently contain compounds with antioxidant activity.

For example, where the biological material used to prepare the particles is obtained from animal lipids and/or plant lipids, an anti-oxidant (e.g. in the form of an extract from rosemary) may be added to the particle.

In the composition, anti-oxidants that may be present in and/or added to the particles include natural material such as plant or microalgal extracts rich in antioxidants (e.g. rosemary or sage extracts containing carnosic acid, green tea extracts containing catechins, Dunaliella salina oleoresins containing carotenoids, spinach raw material or extract containing oxalic acid chelating agent). Compounds present in such plant or microalgal extracts include, but are not limited to, those selected from the group consisting of tocopherols (i.e. α-tocopherol), tocotrienols, plastochromanols, phenolic diterpenes (such as carnosic acid), flavonoids (such as tea catechins), phenolic acids and esters, stilbenes, carotenoids, essential oils (including oxygenated terpenes) and mixtures thereof.

Anti-oxidants that may be added to the particles include synthetic antioxidants selected from the group consisting of butylated hydroxyanisole (BHA), butylated hydroxytoluene (BHT), tert-butyl-hydroxyquinone (TBHQ), propyl gallate (PG), ascorbyl palmitate, ethylenediaminetetraacetic acid (EDTA) and mixtures thereof.

The composition may comprise or consist of particles prepared from plant lipid and/or animal lipid comprising a plant or microalgal extract rich in anti-oxidants, such as anti-oxidants in the form of a rosemary extract.

For example, particles prepared from plant lipid and/or animal lipid may comprise a rosemary extract in the form of a powder, wherein the extract comprises from about 1% to about 70% carnosic acid by weight of the extract or particles prepared from plant lipid and/or animal lipid may comprise a rosemary extract in the form of a liquid comprising from about 1% to about 30% carnosic acid by weight of the composition.

Where the rosemary extract is in the form of a liquid, the rosemary extract may be solubilized and/or suspended in a liquid. Suitable liquids include, but are not limited to, a vegetable oil, such as sunflower oil.

In the composition, the particles may comprise from about 0.01 mg antioxidant/g of particles to about 100 mg antioxidant/g of particles, such as from about 0.1 mg antioxidant/g particle to about 50 mg antioxidant/g particle.

The particles may also be prepared from tripalmitin and/or palm stearin and/or tricaprylin and may optionally comprise anti-oxidants, such as α-tocopherol, carnosic acid or green tea flavonoids.

For example, the composition may comprise or consist of particles prepared from tripalmitin and may optionally further comprise α-tocopherol, or the composition may comprise or consist of particles prepared from palm stearin and may optionally further comprise α-tocopherol.

In some compositions, the particles may optionally not comprise or consist of chitin, protein cages, such as a Bacillus stearothermophilus E2 protein of pyruvate dehydrogenase multi-enzyme complex or an E2LC2 protein, or a gellable hydrophilic polysaccharides, such as agar, agarose, alginates and carrageenans.

The particles used in the composition of the invention may be prepared by:
(i) providing biological material; and
(ii) converting the biological material into particles.

Converting the biological material into particles may comprise removing water from the material (i.e. raw plant material), such as drying the biological material, and then micronizing (e.g. grinding) the biological material into a powder/particles having a particle diameter as previously defined. The biological material may also be milled in water.

Alternatively, the biological material may be extracted with a solvent, such as water or water/alcohol or alcohol or ester or ether or alkane either aromatic or aliphatic, and the solvent removed to yield a solid product, which may optionally be micronized (e.g. ground) into a powder/particles having a particle diameter as previously defined.

Further alternatively, the plant and/or animal and/or microbiological material may be processed to extract high melting point lipids which may then be formed into particles.

For example, when the particles are prepared from high melting point lipids from plants and/or animals, the particles may be prepared by:

a) heating an aqueous phase (for example, heating the aqueous phase to a temperature from about room temperature to 150° C., such as from about 40° C. to about 100° C.);
b) melting a lipid with a high melting point from plants and/or animals;
c) stirring at high speed the product of (a) and (b) to form a coarse emulsion;
d) homogenizing the coarse emulsion obtained at step (c) to obtain a sub-micron emulsion; and
e) cooling down the product of step (d) to allow the lipid phase to crystallize (for example, cooling the product of step (d) to a temperature from about room temperature to about 0° C.).

Optionally, the above method may include incorporating antioxidants into the melted lipid in step (b) and/or drying the preparation obtained in step (e) to provide a solid formulation of particles.

In step (a), the aqueous phase may be heated by such techniques known in the art, such as water bath, heat exchanger, or a tank equipped with heating jacket.

In step (b), the lipid may be melted by such techniques known in the art, such as water bath, heat exchanger, or a tank equipped with heating jacket.

In step (c), the product of (a) and (b) may be stirred by such techniques known in the art, such as rotor-stator homogenisers, ultrasounds, or colloid mills.

In step (d), the coarse emulsion may be homogenized by such techniques known in the art, such as by using high pressure homogenization or ultrasounds to obtain submicron-sized melted fat particles.

In step (e), the product of step (d) may be cooled by such techniques known in the art, such as water bath, heat exchanger, or a tank equipped with heating jacket.

The present invention also provides an emulsion (such as an oil-in-water emulsion or a water-in-oil emulsion) comprising a composition as previously defined, the emulsion comprising an internal phase dispersed in a continuous external phase, wherein particles are located at the interface of the external and the internal phase, and at least one of the internal or external phase comprises an oxidisable compound.

This emulsion is hereinafter referred to as the emulsion of the invention.

As used herein, the term “emulsion” is a liquid product comprising an internal phase dispersed within an external phase for at least 10 minutes, preferably for at least 1 hour, such as at least 24 hours or at least 1 week. As used herein “emulsion” includes single and double emulsions and includes liquid emulsions that comprise a gas internal phase.

As used herein, the term “oxidisable material” means that the material contains functional groups that react with oxygen present in the surrounding environment to form primary and/or secondary oxidation products. The oxidizable material may be a flavour forming compound, a colour forming compound or a mixture thereof.

In the emulsion, the oxidisable material may comprise a lipid, such as a lipid with at least one carbon-carbon unsaturation, such as a double or triple bond in the fatty acyl chain.

For example, the lipid with at least one carbon-carbon double bond in the fatty acyl chain may be selected from the group consisting of palmitoleic acid, oleic acid, myristoleic acid, linoleic acid, arachidonic acid, linolenic acid, eicosapentaenoic acid, docosahexaenoic acid, sunflower (such as stripped sunflower oil), soybean, canola, rapeseed, flaxseed, olive, peanut, corn, cottonseed, palm, and fish oils.

The emulsion may comprise an internal phase comprising oil and an external phase comprising water, hereinafter referred to as an oil-in-water emulsion or may comprise an internal phase comprising water and an external phase comprising oil, hereinafter referred to as a water-in-oil emulsion.

The emulsion may comprise the composition of the invention in an amount from about 0.01% to about 60% by weight of the emulsion, such as from about 1% to about 40%.

The emulsion may comprise from about 1% to about 80% w/w liquid oil, and from about 0.01% to about 60% particles.

The emulsion may be a nutraceutical composition, a dietary or food product for humans or animals (such as functional food compositions, i.e. food, drink, feed or pet food or a food, drink, feed or pet food supplements), a herbicide, a nutritional supplement, a fragrance or flavouring, a pharmaceutical or veterinary composition, an oenological or cosmetic formulation or may form a part of a nutraceutical composition, a dietary or food product for humans or animals (such as functional food compositions, i.e. food, drink, feed or pet food or a food, drink, feed or pet food supplements), a nutritional supplement, a fragrance or flavouring, a pharmaceutical or veterinary composition, an oenological or cosmetic formulation.

For example, the emulsion may be an oil-in-water based sauce such as mayonnaise, hollandaise, béarnaise or salad dressing.

In the emulsion of the invention, the particles located at the interface may completely surround the internal phase or may only partially surround the internal phase. Typically, the particles completely surround the internal phase.

In the emulsion, the diameter of the internal phase is typically no smaller than the diameter of the particles and is preferably larger than particles used. For example, the diameter of the internal phase may be from 0.5 μm to about 1000 μm, such as from about 1 μm to about 500 μm or from about 10 μm to about 100 μm.

The inventors have surprisingly and unexpectedly found that a composition of the invention can provide protection against oxidation of a lipid present in an emulsion.

The present inventors have also found that the presence of an antioxidant in the particles capable of locating at or located at the interface of an emulsion can provide better protection against oxidation of the lipids present in an emulsion than the same particle-stabilized emulsion wherein the particles do not comprise an antioxidant, even when an antioxidant is present in the emulsion, such as where an antioxidant is present in the internal (oil) phase of the emulsion.

For certain applications such as food, pharmaceutical or nutraceutical products, the particles may be edible and/or non-toxic.

If required, emulsifiers may be added in small amount to the emulsion. Typically, the amount of emulsifier added may be less than 5% by weight of the emulsion, such as less than 2% or less than 1% by weight of the emulsion.

The present invention provides a method for reducing or preventing oxidation of an emulsion comprising either:
(i) forming an emulsion comprising an internal phase dispersed in a continuous external phase and then adding a composition as previously defined to the emulsion; or
(ii) forming an emulsion comprising an internal phase dispersed in a continuous external phase and a composition as previously defined by mixing two or more immiscible liquids and the particles under conditions suitable for forming an emulsion;
   wherein at least one of the internal or external phase comprises an oxidisable material.

For example, the present invention provides a method for reducing or preventing oxidation of an emulsion comprising forming an emulsion by mixing a composition as previously defined with either:
(a) two or more immiscible liquids; or
(b) a pre-prepared emulsion comprising an internal phase dispersed in a continuous external phase.

The present invention also provides a method of enhancing the oxidative stability of an emulsion comprising either:
(i) forming an emulsion comprising an internal phase dispersed in a continuous external phase and then adding a composition as previously defined to the emulsion; or
(ii) forming an emulsion comprising an internal phase dispersed in a continuous external phase and a composition as previously defined by mixing two or more immiscible liquids and the particles under conditions suitable for forming an emulsion;
   wherein at least one of the internal or external phase comprises an oxidisable material.

For example, the present invention provides a method for enhancing the oxidative stability of an emulsion comprising forming an emulsion by mixing a composition as previously defined with either:
(a) two or more immiscible liquids; or
(b) a pre-prepared emulsion comprising an internal phase dispersed in a continuous external phase.

The present invention also provides a method of prolonging the shelf-life of a beverage, a nutraceutical, a pharmaceutical or a food product comprising an emulsion, wherein the method comprises either:
(i) forming an emulsion comprising an internal phase dispersed in a continuous external phase and then adding a composition as previously defined to the emulsion; or
(ii) forming an emulsion comprising an internal phase dispersed in a continuous external phase and a composition as previously defined by mixing two or more immiscible liquids and the particles under conditions suitable for forming an emulsion;
   wherein at least one of the internal or external phase comprises an oxidisable material.

For example, the present invention provides a method of prolonging the shelf-life of a beverage, a nutraceutical, a pharmaceutical or a food product comprising an emulsion comprising forming an emulsion by mixing a composition as previously defined with either:
(a) two or more immiscible liquids; or
(b) a pre-prepared emulsion comprising an internal phase dispersed in a continuous external phase.

In the methods described above, the internal phase may comprise oil and the external phase may comprise water or the internal phase may comprise water and the internal phase may comprise oil.

In the methods described above, the composition may be added to the at least two immiscible liquids by:

(i) adding the composition to the at least two immiscible liquids,
(ii) stirring at high speed to form a coarse emulsion.

In the methods described above, the composition may be added to the pre-prepared emulsion by:

(i) adding the composition to the pre-prepared emulsion,
(ii) stirring at high speed to form a coarse emulsion.

For example, where the internal phase is oil and the external phase is water, a composition comprising lipid-based particles as described previously may be added to the emulsion by:
(i) mixing the internal and external phases of the emulsion with the composition comprising lipid-based particles,
(ii) stirring at high speed to form a coarse emulsion.

The emulsion may then optionally be subjected to the following steps:
(iii) homogenizing the coarse emulsion obtained at step (ii)
(iv) cooling down the product of (iii) to allow the lipid phase of the particles to crystallize.

In step (i), the internal and external phases may be mixed by such techniques known in the art, such as mixing tanks.

In the methods described above, typically, the composition of the invention may be added to the at least two immiscible liquids or the pre-prepared emulsion before being homogenized. For example, steps (i) to (iii) are performed in the listed order.

In step (ii), the product of (i) may be mixed by such techniques known in the art, such as high pressure homogenisation, ultrasonication, agitation methods (rotor-stator homogeniser, colloid mill).

The present invention also provides the use of a composition as previously defined to stabilise an emulsion comprising an internal phase dispersed in a continuous external phase by reducing, delaying or preventing oxidation, wherein at least one of the internal or external phase comprises an oxidisable material.

The present invention also provides the use of a composition as previously defined to enhance the oxidative stability of an emulsion comprising an internal phase dispersed in a continuous external phase, wherein at least one of the internal or external phase comprises an oxidisable material.

The present invention also provides the use of a composition as previously defined to prolong the shelf life of a beverage, a nutraceutical, a pharmaceutical or food product comprising an emulsion, wherein the emulsion comprises an internal phase dispersed in a continuous external phase, at least one of the internal or external phase comprises an oxidisable material.

These methods and uses are herein after referred to as the methods and uses of the invention. Typically, in the uses and the methods described above, the composition is capable of locating to, or is located at, an interface between the two or more immiscible liquids.

The oxidisable material may comprise a lipid, such as a lipid with at least one carbon-carbon unsaturation, such as a double or triple bond in the fatty acyl chain.

For example, the lipid may comprise at least one carbon-carbon double bond in the fatty acyl chain is selected from the group consisting of palmitoleic acid, oleic acid, myristoleic acid, linoleic acid, arachidonic acid, linolenic acid, eicosapentaenoic acid, docosahexaenoic acid, sunflower, soybean, canola, rapeseed, flaxseed, olive, peanut, corn, cottonseed, palm, and fish oils.

The emulsion may comprise an internal phase comprising oil and an external phase comprising water, hereinafter referred to as an oil-in-water emulsion.

The emulsion may be or may form part of a nutraceutical composition, a dietary or food product for humans or animals (such as functional food compositions, i.e. food, drink, feed or pet food or a food, drink, feed or pet food supplements), a herbicide, a nutritional supplement, a fragrance or flavourings, a pharmaceutical or veterinary composition, an oenological or cosmetic formulation.

For example, the emulsion may be a food that comprises an oil-in-water emulsion or which is an oil-in-water emulsion, such as an egg and oil-based sauce, e.g. mayonnaise, hollandaise or béarnaise or salad dressing.

The presence of the particles in the emulsion reduces, delays and/or prevents the formation of oxidation products such as primary oxidation products including lipid hydroperoxides and conjugated diene hydroperoxides and/or secondary oxidation products including aldehyde, ketone, alcohol, and carboxylic acid volatile compounds as well as non-volatile secondary oxidation products such as p-anisidine, epoxides, dimers and polymers.

The composition comprising particles may be present in the emulsion in an amount from about 0.01% to about 60% by weight of the emulsion, such as from about 1% to about 40%.

The present invention provides a nutraceutical composition, a dietary or food product for humans or animals (such as functional food compositions, i.e. food, drink, feed or pet food or a food, drink, feed or pet food supplements), a herbicide, a nutritional supplement, fragrance or flavouring, a pharmaceutical or veterinary composition, an oenological or cosmetic formulation comprising a composition of the invention and/or an emulsion of the invention.

The present invention also provides the use the composition of the invention and/or the emulsion of the invention in a nutraceutical composition, a dietary or food product for humans or animals (such as functional food compositions, i.e. food, drink, feed or pet food or a food, drink, feed or pet food supplements), a herbicide, a nutritional supplement, a fragrance or flavouring, a pharmaceutical or veterinary composition, an oenological or cosmetic formulation.

The nutraceutical compositions, dietary or food products for humans or animals (such as functional food compositions, i.e. food, drink, feed or pet food or a food, drink, feed or pet food supplements), nutritional supplements, fragrances or flavourings, pharmaceutical or veterinary compositions, oenological or cosmetic formulations may optionally further comprise a pharmaceutically/veterinary ingredients, such as excipients or carriers or (function) food acceptable ingredients and mixtures thereof as appropriate.

The nutraceutical compositions, dietary or food products for humans or animals (such as functional food compositions, i.e. food, drink, feed or pet food or a food, drink, feed or pet food supplements), herbicide, nutritional supplements, fragrances or flavourings, pharmaceutical or veterinary compositions, oenological or cosmetic formulations may consist of or consist essentially of the emulsion of the invention.

For the avoidance of doubt, in this specification when we use the term “comprising” or “comprises” we mean that the extract or composition being described must contain the listed ingredient(s) but may optionally contain additional ingredients. When we use the term “consisting essentially of” or “consists essentially of” we mean that the extract or composition being described must contain the listed ingredient(s) and may also contain small (for example up to 5% by weight, or up to 1% or 0.1% by weight) of other ingredients provided that any additional ingredients do not affect the essential properties of the extract or composition. When we use the term “consisting of” or “consists of” we mean that the extract or composition being described must contain the listed ingredient(s) only.

It is also intended that the terms “comprise” or “comprises” or “comprising” may be replaced with “consist” or “consisting” or “consisting” throughout the application.

As used herein, references to pharmaceutically or veterinary acceptable excipients may refer to pharmaceutically or veterinary acceptable adjuvants, diluents and/or carriers as known to those skilled in the art.

Food acceptable ingredients include those known in the art (including those also referred to herein as pharmaceutically acceptable excipients) and can be natural or non-natural, i.e. their structure may occur in nature or not. In certain instances, they can originate from natural compounds and be modified before use (e.g. maltodextrin).

By “pharmaceutically or veterinary acceptable” we mean that the additional components of the composition are generally safe, non-toxic, and neither biologically nor otherwise undesirable. For example, the additional components are generally sterile and pyrogen free. Such components must be “acceptable” in the sense of being compatible with the emulsion of the invention and not deleterious to the recipients thereof. Thus, “pharmaceutically acceptable excipients” includes any compound(s) used in forming a part of the formulation that is intended to act merely as an excipient, i.e. not intended to have biological activity itself.

The skilled person will understand that compositions comprising a composition of the invention and/or an emulsion of the invention (e.g. in the form of compositions, such as pharmaceutical or veterinary compositions) may be administered to a patient or subject (e.g. a human or animal patient or subject) by any suitable route, such as by the oral, rectal, nasal, pulmonary, buccal, sublingual, transdermal, intracisternal, intraperitoneal, or parenteral (including subcutaneous, intramuscular, intrathecal, intravenous and intradermal) route.

Compositions (e.g. pharmaceutical or veterinary or food compositions) comprising a composition of the invention and/or an emulsion of the invention may be administered orally. In such instances, pharmaceutical or veterinary compositions according to the present invention may be specifically formulated for administration by the oral route.

Liquid dosage forms for oral administration include solutions, emulsions, aqueous or oily suspensions, syrups and elixirs.

Compositions (e.g. pharmaceutical or veterinary or food compositions) described herein, such as those intended for oral administration, may be prepared according to methods known to those skilled in the art, such as by mixing the components of the composition together.

The compositions (e.g. pharmaceutical or veterinary or food compositions) may contain one or more additional ingredients, such as food ingredients or pharmaceutical ingredients and excipients, such as sweetening agents, flavouring agents, colouring agents and preserving agents. The compositions of the invention may contain the active ingredient(s) in admixture with non-toxic pharmaceutically acceptable excipients (or ingredients). These excipients (or ingredients) may, for example, be: inert diluents, such as calcium carbonate, sodium carbonate, lactose, calcium phosphate or sodium phosphate; granulating and disintegrating agents, for example, corn starch, maltodextrin or alginic acid; binding agents, for example, starch, gelatine or acacia; or lubricating agents, for example magnesium stearate, stearic acid, talc and mixtures thereof.

Liquid compositions (e.g. pharmaceutical or veterinary or food compositions) may be contained within a capsule, which may be uncoated or coated as defined above.

Suitable pharmaceutical or veterinary carriers include inert solid diluents or fillers, sterile aqueous solutions and various organic solvents. Examples of liquid carriers are syrup, peanut oil, olive oil, phospholipids, fatty acids, fatty acid amines, polyoxyethylene and water.

Moreover, the carrier or diluent may include any sustained release material known in the art, such as glyceryl monostearate or glyceryl distearate, alone or mixed with a wax.

Suitable pharmaceutical carriers include inert sterile aqueous solutions and various organic solvents. Examples of liquid carriers are syrup, vegetables oils, phospholipids, fatty acids, fatty acid amines, polyoxyethylene and water. Moreover, the carrier or diluent may include any sustained release material known in the art, such as glyceryl monostearate or glyceryl distearate, alone or mixed with a wax.

The term “carrier” as used herein, may also refer to a natural product or a product originating from nature that has been transformed or modified so that it is distinct from the natural product from which it originated, such as maltodextrin.

For pharmaceutical and/or veterinary products, depending on the disorder and the subject to be treated, as well as the route of administration, compositions comprising or consisting of the emulsion of the invention may be administered at varying doses (i.e. therapeutically effective doses, as administered to a patient in need thereof). In this regard, the skilled person will appreciate that the dose administered to a mammal, particularly a human, in the context of the present invention should be sufficient to affect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the age, condition, body weight, sex and response of the patient to be treated, and the stage/severity of the disease.

The pharmaceutical or veterinary compositions comprising a composition of the invention and/or an emulsion of the invention in a therapeutically effective amount. As used herein, the term “effective amount” is synonymous with “therapeutically effective amount”, “effective dose”, or “therapeutically effective dose” and when used in the present invention refers to the minimum dose of the emulsion of the invention necessary to achieve the desired therapeutic effect and includes a dose sufficient to reduce a symptom associated with inflammation. Effectiveness in treating the diseases or conditions described herein can be determined by observing an improvement in an individual based upon one or more clinical symptoms, and/or physiological indicators associated with the condition. An improvement in the diseases or conditions described herein also can be indicated by a reduced need for a concurrent therapy.

Additionally, where repeated administration of the emulsion of the invention is used, an effective amount of the emulsion of the invention will further depend upon factors, including, without limitation, the frequency of administration, the half-life of the extract of the invention, or any combination thereof.

The amount of the composition of the invention and/or an emulsion of the invention present in nutraceutical compositions, dietary or food products for humans or animals (such as functional food compositions, i.e. food, drink, feed or pet food or a food, drink, feed or pet food supplements), nutritional supplements, fragrances or flavourings, pharmaceuticals (pharmaceutical compositions or formulations), veterinary compositions, oenological or cosmetic formulations will vary depending on the application.

Typically, the amount of composition of the invention and/or an emulsion of the invention present in nutraceutical compositions, dietary or food products for humans or animals (such as functional food compositions, i.e. food, drink, feed or pet food or a food, drink, feed or pet food supplements), herbicide, nutritional supplements, fragrances or flavourings, pharmaceuticals (pharmaceutical compositions or formulations), veterinary compositions, oenological or cosmetic formulations will be from about 0.001 to about 50% by weight, such as from about 0.01% to about 30% or from about 1% to about 20% of the nutraceutical compositions, dietary or food product, herbicide, nutritional supplement, fragrance or flavouring, pharmaceutical composition or formulation, veterinary composition, oenological or cosmetic formulation, such as from about 0.01 to about 20%, or from about 0.1 to 10% or from about 1 to about 5% by weight of the formulation.

The emulsions of the invention are suitable for use in a wide range of food products. Such food products include, but are not limited to, raw meat products, cooked meat products, raw poultry products, cooked poultry products, raw seafood products, cooked seafood products, ready to eat meals, cooking sauces, such as pasta sauces and ketchups, table sauces, pasteurised and unpasteurised soups, salad dressings and other oil-in-water emulsions e.g. mayonnaise, water-in-oil emulsions, dairy products, bakery products, confectionary products, fruit products and foods with fat based or water containing filings. Preferably, the foodstuff comprises an oil-in-water-emulsion or is an oil-in-water emulsion. For example, the foodstuff may be a table sauce, such as an egg and oil-based sauce, e.g. mayonnaise, hollandaise or béarnaise or a foodstuff comprising a table sauce, such as an egg and oil-based sauce, e.g. mayonnaise, hollandaise or béarnaise.

The food product typically contains composition or the emulsion of the invention in an amount sufficient to stabilise the food product, for example, to reduce, delay, inhibit or prevent oxidation of the food product. Typically, the stabilising composition is present in the foodstuff in an amount from about 0.1% to about 20% by weight of the foodstuff. Such as from about 0.5% to about 10%, or from about 1% to about 5% or 2.5%.

When the composition or the emulsion of the invention is incorporated into a food product or is a food product, such as egg and oil based sauces, e.g. mayonnaise, hollandaise or béarnaise, as well as providing a stabilising effect by reducing, inhibiting or preventing the amount of oxidation over a given period relative to the amount of oxidation that would have occurred in the absence of the emulsion, the emulsion of the invention should not adversely affect the colour of the food product in which it has been incorporated.

The present invention provides a method for the preparation of an emulsion as previously defined, wherein the method comprises:
mixing a composition as previously defined with either:
   (a) two or more immiscible liquids; or
   (b) a pre-prepared emulsion comprising an internal phase dispersed in a continuous external phase.

This method is hereinafter referred to as the preparation method of the invention.

The internal phase may comprise oil and the external phase may comprise water or the internal phase may comprise water and the internal phase may comprise oil.

In the preparation method, the composition may be added to the at least two immiscible liquids by:

(i) adding the composition to the at least two immiscible liquids,
(ii) stirring at high speed to form a coarse emulsion.

In the preparation method, the composition may be added to the pre-prepared emulsion by:

(i) adding the composition to the pre-prepared emulsion,
(ii) stirring at high speed to form a coarse emulsion.

For example, where the internal phase is oil and the external phase is water, a composition comprising lipid-based particles as described previously may be added to the emulsion by:
(i) mixing the internal and external phases of the emulsion with the composition comprising lipid-based particles,
(ii) stirring at high speed to form a coarse emulsion.

The emulsion may then optionally be subjected to the following steps:
(iii) homogenizing the coarse emulsion obtained at step (ii)
(iv) cooling down the product of (iii) to allow the lipid phase of the particles to crystallize.

In step (i), the internal and external phases may be mixed by such techniques known in the art, such as mixing tanks.

In the preparation method, typically, the composition of the invention may be added to the at least two immiscible liquids or the pre-prepared emulsion before being homogenized. For example, steps (i) to (iii) are performed in the listed order.

In step (ii), the product of (i) may be mixed by such techniques known in the art, such as high pressure homogenisation, ultrasonication, agitation methods (rotor-stator homogeniser, colloid mill).

In step (iii), the coarse emulsion may be homogenized by such techniques known in the art, such as using high pressure to obtain submicron-sized melted fat droplet.

In step (iv), the product of step (iii) may be cooled by such techniques known in the art, such as water bath, heat exchanger, thermostatic tanks (heating jacket).

The present invention also provides a kit for use to stabilise an emulsion comprising an internal phase dispersed in a continuous external phase by reducing, delaying or preventing oxidation, wherein at least one of the internal or external phase comprises an oxidisable material; the kit comprising particles as defined above and instructions for use.

The present invention also provides a kit for use to enhance the oxidative stability of an emulsion comprising an internal phase dispersed in a continuous external phase, wherein at least one of the internal or external phase comprises an oxidisable material; the kit comprising particles as defined above and instructions for use.

The present invention also provides a kit for use to prolong the shelf life of a beverage, a nutraceutical, a pharmaceutical or food product comprising an emulsion, wherein the emulsion comprises an internal phase dispersed in a continuous external phase, and at least one of the internal or external phase comprises an oxidisable material; the kit comprising particles as defined above and instructions for use.

### BRIEF DESCRIPTION OF THE FIGURES

FIG. 1. Schematic representation of an oil-in-water emulsion stabilized by tripalmitin colloidal particles (A) or a conventional emulsifier where tripalmitin is solubilised in the oil phase (B).

FIG. 2. Lipid oxidation kinetics measured in conventional sodium caseinate-stabilized emulsion containing tripalmitin fat (circles) and Pickering emulsion stabilized by tripalmitin colloidal particles (squares). Both emulsions are incubated with 200 μM FeSO₄/EDTA at 25° C. CD, conjugated dienes (left); p-AV, p-anisidine (right).

FIG. 3. Schematic representation of an oil-in-water emulsion stabilized by palm stearin colloidal particles (A) or stabilized by a conventional emulsifier where palm stearin is solubilised in the oil phase (B).

FIG. 4. Lipid oxidation kinetics measured in conventional sodium caseinate-stabilized emulsion containing palm stearin fat (circles) and Pickering emulsion stabilized by palm stearin colloidal particles (squares). Both emulsions are incubated with 200 μM FeSO₄/EDTA at 25° C. CD, conjugated dienes (left); p-AV, p-anisidine (right).

FIG. 5. Characterization of the particle size distribution of some representative natural powders suspended in water at 1% (w/w). Matcha tea raw material (A), spinach leave raw material (B), spirulina extraction cake (C), pineapple fibers (D), and rosemary leave extraction cake (E). Non-micronized powder: solid line; micronized powder: dotted line.

FIG. 6. Scanning electron micrographs of the non-micronized and micronized (respectively) powders of matcha tea raw material (A and B), pineapple fibers (C and D), spinach raw material (E and F), rosemary leaves extraction cakes (G and H), spirulina extraction cakes (I and J), curcuma extract (K and L), and red radish extract (M and N).

FIG. 7. Particle size distribution of oil-in-water emulsions stabilized during three months at 4° C. by non-micronized natural powders or conventional emulsifiers. The tested powders are spinach (A), spirulina (B), matcha tea (C), pineapple fibers (D), while the conventional emulsifiers are Tween 60 at 1% (w/w) (E), egg yolk at 5% (w/w) (F). t₀: solid line; t₃: dotted line. All emulsions were prepared in a 50 mM acetate buffer pH 4.5 and contained 0.1 wt % potassium sorbate as antimicrobial.

FIG. 8. Particle size distribution of Pickering oil-in-water emulsions stabilized during one month at 4° C. by 5% wt non-micronized natural particles and added with 100 mM NaCl. Matcha tea (A), spinach leaves (B), and spirulina cakes (C). t₀: solid line; t₁: dotted line. All emulsions were prepared in a 50 mM acetate buffer pH 4.5.

FIG. 9. Particle size distribution of Pickering oil-in-water emulsions stabilized during one month at 4° C. by 5% wt non-micronized natural particles and added with 100 mM NaCl. Matcha tea (A), spinach leaves (B), spirulina cakes (C), and pineapple fibers (D). t₀: solid line; t₁: dotted line. All emulsions were prepared in a 50 mM phosphate buffer pH 7.0.

FIG. 10. Particle size distribution of Pickering oil-in-water emulsions stabilized during one month at 4° C. by 5% wt non-micronized pineapple fibers and added with 100 mM NaCl. t₀: solid line; t₁: dotted line. All emulsions were prepared in unbuffered ultrapure water of “Type 1” as defined by ISO3696 (for example, milliQ water).

FIG. 11. Particle size distribution of Pickering oil-in-water emulsions stabilized during one month at 4° C. by 5% wt non-micronized natural particles at acidic and neutral pH. Matcha tea at pH 4.5 (A) and pH 7.0 (B), and spirulina cakes at pH 4.5 (C) and pH 7.0 (D). t₀: solid line; t₁: dotted line. Emulsions at pH 4.5 were prepared in a 50 mM acetate buffer, while those at pH 7.0 were in a 50 mM phosphate buffer.

FIG. 12. Lipid oxidation kinetics measured in a conventional Tween 60-stabilized oil-in-water emulsion and in a Pickering oil-in-water emulsion stabilized by 5% (w/w) of a non-micronized spirulina cake powder. All emulsions are incubated at 25° C. for four months. All emulsions are prepared with a stripped sunflower oil and a 50 mM acetate buffer pH 4.5. They contain 0.1 wt % potassium sorbate as antimicrobial.

FIG. 13. Lipid oxidation kinetics measured in a conventional egg yolk-stabilized oil-in-water emulsion and in two Pickering oil-in-water emulsions stabilized by 5% (w/w) of a non-micronized matcha tea powder or 5% (w/w) of a non-micronized spinach leave powder. All emulsions are incubated at 25° C. for four months. All emulsions are prepared with a stripped sunflower oil and a 50 mM acetate buffer pH 4.5. They contain 0.1 wt % potassium sorbate as antimicrobial.

FIG. 14. Representative micrographs of Pickering water-in-oil emulsions (reverse emulsions) stabilized by 1% (w/w) non-micronized curcuma extract (A) and 2.5% (w/w) non-micronized rosemary leave extraction cakes (B).

FIG. 15. Surface-activity of the supernatants obtained after applying a washing procedure to the natural particles.

FIG. 16. Particle size distribution of oil-in-water emulsions stabilized during one week at 4° C. by 5% wt of the supernatant of washed non-micronized natural particles. Matcha tea (A), spinach leaves (B), spirulina cakes (C), and pineapple fibers (D). t₀: solid line; t₁: dotted line. All emulsions are prepared in a 50 mM acetate buffer pH 4.5.

FIG. 17. Particle size distribution of Pickering oil-in-water emulsions stabilized during four weeks (excepted for pineapple, 1 week) at 4° C. by 5% wt of washed non-micronized natural particles. Matcha tea (A), spinach leaves (B), spirulina cakes (C), and pineapple fibers (D). t₀: solid line; t₄: dotted line. All emulsions are prepared in a 50 mM acetate buffer pH 4.5.

FIG. 18. Schematic representation of two types of colloidal particle-stabilized stripped sunflower oil-in-water emulsions (Pickering emulsions). Emulsion composition is identical, but α-tocopherol is incorporated in the colloidal particles (the emulsion of the invention) (A) or in the liquid PUFA oil droplets (control emulsion) (B). Conjugated diene hydroperoxide (CD-LOOH) concentration (C), p-anisidine value (p-AV) (D) and α-tocopherol degradation (E) in both oil-in-water emulsions incubated at 25° C. with 200 μM FeSO₄/EDTA. Averaged values±standard deviations result from independent triplicates. Symbols: the emulsion of the invention (A); square grey symbols: the control emulsion (B) black circles. Both Pickering emulsions are stabilized by tripalmitin colloidal particles.

FIG. 19. Confocal laser scanning microscopy images of the Pickering stripped sunflower oil-in-water emulsion of the invention (A) and of the control emulsion (B) with 25-NBD-cholesterol (fluorescent analogue of α-tocopherol) initially added in tripalmitin colloidal particles (A) or within the droplets (B), taken at different time points. Polarized light microscopy images of the Pickering emulsion of the invention produced with colloid mill homogenization at t₀ and t_{72 h} (C and D, respectively). In panels A and B, the scale bar represents 10 μm. Both Pickering emulsions are stabilized by tripalmitin colloidal particles.

FIG. 20. DSC melting and crystallization thermograms of the Pickering stripped sunflower oil-in-water emulsion of the invention stabilized by tripalmitin colloidal particles containing α-tocopherol at t₀ and t₃₃₆ (C).

FIG. 21. Schematic representation of two types of colloidal particle-stabilized stripped sunflower oil-in-water emulsions (Pickering emulsions). Emulsion composition is identical, but carnosic acid is incorporated in the tripalmitin colloidal particles (the emulsion of the invention) (A) or in the liquid PUFA oil droplets (control emulsion) (B). Conjugated diene hydroperoxide (CD-LOOH) concentration (C) and p-anisidine value (p-AV) (D) in both oil-in-water emulsions incubated at 25° C. with 200 μM FeSO₄/EDTA. Averaged values±standard deviations result from independent triplicates. Symbols: the emulsion of the invention (A); square grey symbols: the control emulsion (B) black circles.

FIG. 22. Schematic illustration of two types of conventional sodium caseinate-stabilized stripped sunflower oil-in-water emulsions comprising a suspension of tripalmitin colloidal particles in their aqueous phase. Emulsion and suspension composition is identical, but antioxidant is located either in the suspended tripalmitin colloidal particles (A) or in the core of the oil droplets (B). Total antioxidant concentration is similar in both systems. Conjugated diene hydroperoxide (CD-LOOH) content (C), p-anisidine value (p-AV) (D), and α-tocopherol recovery (E) of the two types of emulsions with α-tocopherol in the suspended colloidal particles (black circles) or in the liquid oil droplets (grey squares), incubated with 200 μM FeSO₄/EDTA at 25° C. Averaged values+/−standard deviation result from independent triplicates (C, D, and E).

FIG. 23. Confocal laser scanning microscopy images of the conventional stripped sunflower oil-in-water emulsion with added tripalmitin colloidal particles with 25-NBD-cholesterol (fluorescent analogue of α-tocopherol) initially added in the palmitin colloidal particles (A) or in the droplets (B), taken at different time points. In panels A and B, the scale bar represents 10 μm.

FIG. 24. Schematic illustration of Pickering stripped sunflower oil-in-water emulsions comprising an interfacially-adsorbed population of antioxidant-free tripalmitin colloidal particles and an aqueous phase-suspended population of tripalmitin colloidal particles containing α-tocopherol (A) or not (B). Emulsion and suspension composition is identical, but antioxidant is located either in the suspended colloidal particles (A) or in the core of the oil droplets (B). Total antioxidant concentration is similar in both systems. Conjugated diene hydroperoxide (CD-LOOH) content (C), p-anisidine value (p-AV) (D), and a-tocopherol recovery (E) of the two types of emulsions with α-tocopherol in the suspended colloidal particles (black circles) or in the liquid oil droplets (grey squares), incubated with 200 μM FeSO₄/EDTA at 25° C. Averaged values+/−standard deviation result from independent triplicates (C, D, and E).

FIG. 25. Confocal laser scanning microscopy images taken at different time points of the Pickering stripped sunflower oil-in-water emulsions comprising an interfacially-adsorbed population of antioxidant-free palmitin colloidal particles and an aqueous phase-suspended population of tripalmitin colloidal particles containing 25-NBD-cholesterol (fluorescent analogue of α-tocopherol, A) or not (B). In this latter case, the fluorescent analogue is initially located in the oil droplets. In panels A and B, the scale bar represents 10 μm.

FIG. 26. Conjugated diene hydroperoxide (CD-LOOH) concentration (A), p-anisidine value (p-AV) (B), and α-tocopherol degradation (C) during incubation of the Pickering stripped sunflower oil-in-water emulsion of the invention containing 90 (black circles), 45 (triangles) or 22.5 (diamonds) ppm of α-tocopherol in the tripalmitin colloidal particles, and a control Pickering oil-in-water emulsion containing 90 ppm of α-tocopherol in the oil droplets (grey squares).

FIG. 27. Characterization of tripalmitin colloidal particles with or without α-tocopherol. Particle size distribution (A), DSC melting and crystallization thermograms (B), and TEM image of tripalmitin colloidal particles with tocopherol (C).

FIG. 28. Characterization of Pickering stripped sunflower oil-in-water emulsions with a-tocopherol either in the tripalmitin colloidal particles (the emulsion of the invention) or in the core of the oil droplets (control emulsion). Droplet size distribution (A), DSC melting and crystallization thermogram (B) and TEM image (C) of the emulsion of the invention.

FIG. 29. Stability of α-tocopherol during incubation of Pickering oil-in-water emulsions containing medium chain triglycerides with α-tocopherol either in the tripalmitin colloidal particles (black circles) or in the core of the oil droplets (grey squares).

FIG. 30. DSC melting and crystallization thermograms of a conventional sodium caseinate-stabilized stripped sunflower oil-in-water emulsion comprising tripalmitin colloidal particles in the aqueous phase (solid line), and a tripalmitin colloidal particle dispersion (dashed line).

FIG. 31. Schematic representation of two types of colloidal particle-stabilized stripped sunflower oil-in-water emulsions (Pickering emulsions). Emulsion composition is identical, but α-tocopherol is incorporated in the particles (the emulsion of the invention) (A) or in the liquid PUFA oil droplets (control emulsion) (B). Conjugated diene hydroperoxide (CD-LOOH) concentration (C), p-anisidine value (p-AV) (D) and α-tocopherol degradation (E) in both oil-in-water emulsions incubated at 25° C. with 200 μM FeSO₄/EDTA. Averaged values±standard deviations result from independent triplicates. Symbols: the emulsion of the invention (A); grey squares: the control emulsion (B) black circles. Both Pickering emulsions are stabilized by tripalmitin (80%) colloidal particles containing 20% (w/w) of liquid tricaprylin.

FIG. 32. Confocal laser scanning microscopy images of the Pickering stripped sunflower oil-in-water emulsion of the invention (A) and of the control emulsion (B) with 25-NBD-cholesterol (fluorescent analogue of α-tocopherol) initially added in colloidal particles (A) or within the droplets (B), taken at different time points. Polarized light microscopy images of the Pickering emulsion of the invention produced with colloid mill homogenization at t₀ and t_{72 h} (C and D, respectively). In panels A and B, the scale bar represents 10 μm. Both Pickering emulsions are stabilized by tripalmitin colloidal particles.

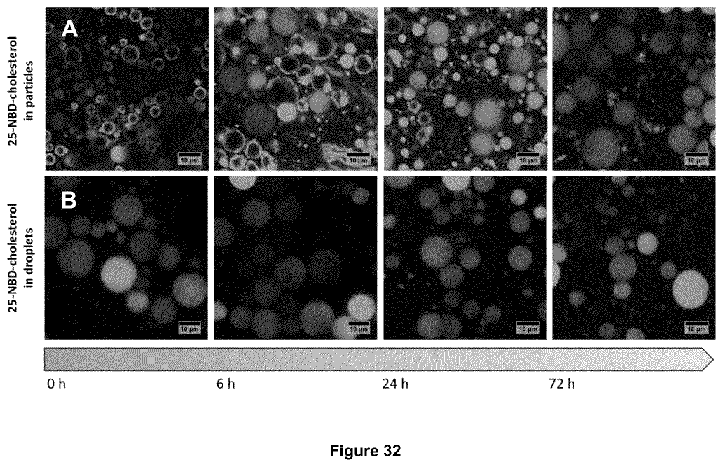

FIG. 33. Schematic representation of two types of colloidal particle-stabilized non stripped flaxseed oil-in-water emulsions (Pickering emulsions). Emulsion composition is identical, but α-tocopherol is incorporated in the particles (the emulsion of the invention) (A) or in the liquid PUFA oil droplets (control emulsion) (B). Conjugated diene hydroperoxide (CD-LOOH) concentration (C) in both oil-in-water emulsions incubated at 25° C. with 200 μM FeSO₄/EDTA. Averaged values±standard deviations result from independent triplicates. Symbols: the emulsion of the invention (A); grey squares: the control emulsion (B) black circles. Both Pickering emulsions are stabilized by tripalmitin colloidal particles.

### EXAMPLES

The present invention will be further described by reference to the following, non-limiting examples.

### Material and Methods

1) Materials

Tripalmitin (#T8127, purity >99%), sodium phosphate monobasic (#S9638), sodium phosphate dibasic (#S9763), sodium chloride (#S7653), iron(II) sulfate heptahydrate (#F8633), ethylenediaminetetraacetic acid disodium salt dihydrate (#E6635), para-anisidine (#A88255), and acetic acid (#45726) were purchased from Sigma-Aldrich. N-Hexane (#808023502) was obtained from Actu-All Chemicals (Oss, the Netherlands). 2-Propanol was purchased from Merck (Darmstadt, Germany). Sodium caseinate was supplied by DMV International (#41610, spray dried, protein content 91.0%). Sunflower oil was obtained from a local supermarket, and was stripped with alumina powder (MP EcoChrome™ ALUMINA N, Activity: Super I, Biomedicals) to remove impurities and tocopherols. Palm stearin (palmitic acid, 82%; oleic acid, 9%; stearic acid, 5%) was supplied by ADM (Saint Laurent Bangy, France). Ultrapure water (18.2 MΩ) was used for all experiments, and was prepared using a Milli-Q system (Millipore Corporation, Billerica, Mass., USA). All other chemicals or solvents were of analytical grade.

2) Purification of Tripalmitin

Tripalmitin was purified by three recrystallization steps using ethanol. Briefly, tripalmitin was dissolved in ethanol at 60-70° C. while stirring for 15 min and left to cool down to room temperature to allow recrystallization, after which ethanol was removed, which was repeated two more times.

3) Preparation of Colloidal Lipid Particles (CLPs)

An aqueous phase containing sodium caseinate in phosphate buffer (10 mM, pH 7.0) was heated in a water bath and added to a melted fat phase (tripalmitin, palm stearin or tricaprylin).

When the particles contained tocopherol, 100 μL α-tocopherol prepared in methanol (200 mg mL⁻¹) was added at this stage. Final α-tocopherol concentrations were 4 mg mg⁻¹ of fat.

A coarse emulsion was then prepared by high speed stirring.

The coarse emulsion was then homogenized at high pressure and temperature then left to cool down, allowing for the lipid phase to crystallize.

4) A General Procedure for the Preparation of O/W Emulsions for Studying the Antioxidative Effect of Particles Prepared from One or More Biological Materials

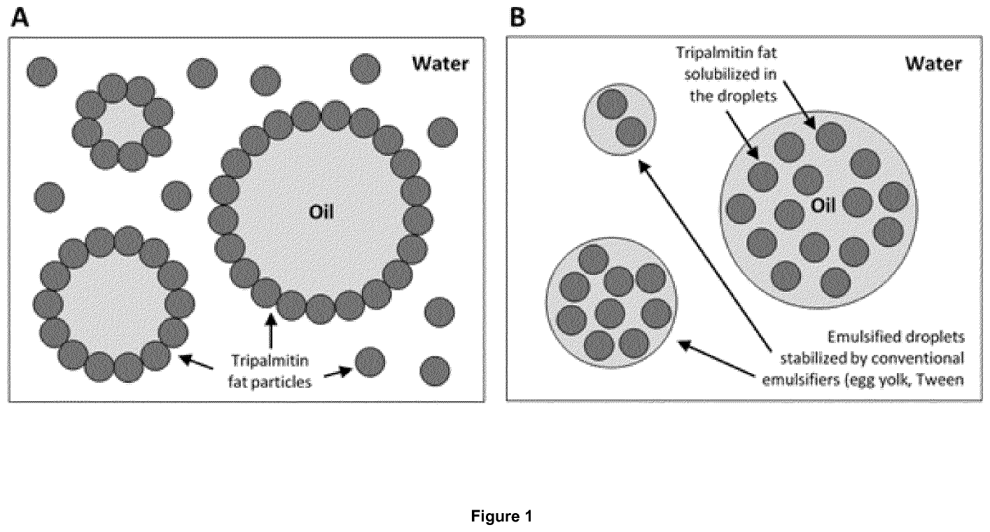

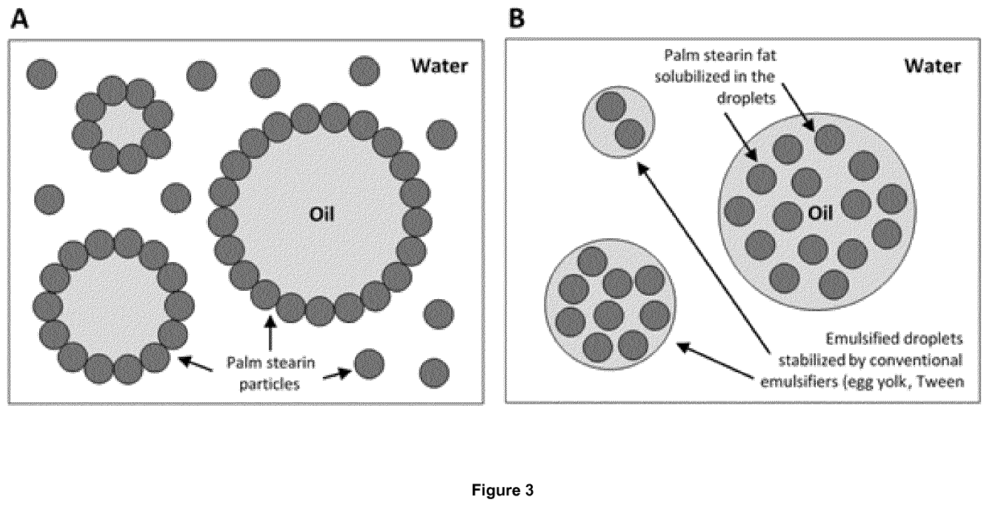

Two types of oil in water emulsions were prepared: one Pickering emulsion, stabilized by colloidal lipid particles (CLP) (tripalmitin or palm stearin) as the one or more biological materials (FIGS. 1A and 3A); and a conventional sodium caseinate-stabilized oil-in-water emulsion containing the same HMP fat (tripalmitin or palm stearin) in its oil interior (FIGS. 1B and 3B). Both emulsions contained the same amount of HMP fat but differed in their structural organization.

For the conventional sodium caseinate-stabilized emulsion containing HMP fat, stripped sunflower oil was mixed with tripalmitin, phosphate buffer (10 mM, pH 7.0) and sodium caseinate in phosphate buffer (10 mM, pH 7.0) at elevated temperature.

For the CLP-stabilized Pickering emulsion, stripped sunflower oil was mixed with phosphate buffer (10 mM, pH 7.0) and a particle dispersion.

The O/W emulsions were processed either by high pressure homogenization or colloid mill homogenization.

Coarse emulsions were prepared by high speed stirring. The obtained emulsions were then either homogenized at high pressure or processed through a colloid mill.

5) A General Procedure for the Preparation of O/W Emulsions for Studying the Antioxidative Effect of Particles Prepared from One or More Biological Materials Filled with an Antioxidant

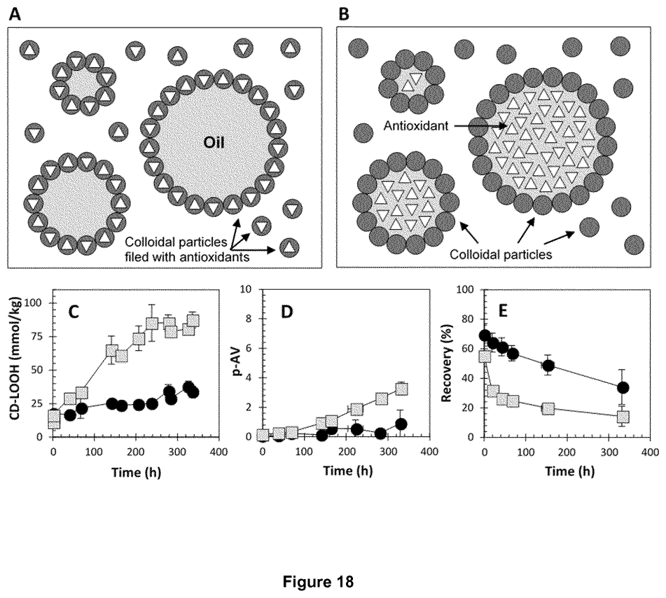

Two types of oil in water Pickering emulsions were prepared; one with α-tocopherol in the particles, and one with α-tocopherol in the liquid sunflower oil droplets (FIGS. 18, 21, 31, and 33).

In the former case, sunflower oil, preliminary stripped from surface-active impurities, was mixed with phosphate buffer (10 mM, pH 7.0) and a particle dispersion (with α-tocopherol in the particles).

In the latter case, components were mixed in the same proportions, but the particles did not contain α-tocopherol, whereas the sunflower oil was added with 100 μL α-tocopherol prepared in methanol (200 mg mL⁻¹), before homogenization.

The mixtures were processed by high speed stirring. The obtained emulsions were then homogenized at high pressure and stored at cold temperature.

6) Extraction and Analysis of α-Tocopherol

α-Tocopherol was extracted from CLPs dispersions or emulsions. First, 4 mL chloroform, 3 mL methanol and 1 mL saturated sodium chloride solution were added to 2 mL of CLP dispersion or emulsion in a 15-mL polypropylene centrifugation tube, which were vortexed followed by centrifugation at 3000×g for 10 minutes. The clear chloroform phase was then collected by cautiously boring a hole in the bottom of the centrifugation tube.

Extracts were analysed on a UltiMate 3000 liquid chromatography system (Thermo Scientific, Sunnyvale, Calif., USA) using a C30 reversed phase column, 3 μm, 150×4.6 mm (YMC, Dinslaken, Germany). Extracts were eluted at 1 mL min′ at 30° C. using a mobile phase with a linear gradient going from 81% methanol, 14% methyl t-butyl ether (MTBE) and 4% Milli-Q water to 74% methanol, 22% methyl t-butyl ether and 4% Milli-Q water in 8 minutes, and going back to its initial composition in 2 minutes. α-Tocopherol was detected with a UV-VIS detector at 292 nm (Dionex™ UltiMate™ 3000 Variable Wavelength Detector), and contents were calculated using a calibration curve that was linear in the range from 5 μg mL⁻¹ to 5000 μg mL⁻¹. The recovery (Rec %) of α-tocopherol in CLPs was calculated as:

$$Rec \% = 100 \frac{{C}_{ex}}{{C}_{in}}$$

where Cₑₓ is the content of extracted α-tocopherol and Cᵢₙ the content of initially added a-tocopherol.

7) Lipid Oxidation Experiments

A catalyst consisting of an equimolar mixture of FeSO₄ and EDTA was prepared by separately dissolving FeSO₄ and EDTA (12 mM) in ultrapure water. Equivalent volumes of each solution were mixed, and the iron-EDTA complex was allowed to form under moderate stirring for 1 h in the dark (Berton, Ropers, Viau, & Genot, 2011). Aliquots of emulsion (2 g) were distributed in a 15-mL polypropylene centrifugation tube. The catalyst (100 μL) was added to the emulsions to obtain a final concentration of 200 μM of both iron and EDTA. The tubes were rotated in the dark at 2 rpm at 25° C. for 0 to 72 h.

### Formation of Conjugated Diene Hydroperoxides (CD-LOOH).

Quantification of CD-LOOH, which are primary lipid oxidation products, was adapted from Corongiu & Banni (1994). In short, the incubated emulsions were diluted 4000-fold in 2-propanol in multiple steps. The final solutions were centrifuged at 20238×g for 1 minute (Centrifuge 5424, Eppendorf Hamburg, Germany), and the absorbance of the supernatant was measured at 233 nm with a UV-visible spectrophotometer (DU 720 Beckman Coulter, Brea, Calif., USA). The reference cell contained 2-propanol and phosphate buffer (10 mM, pH 7.0) in the same proportions as in the final dilution of the samples. Results were expressed in mmol of equivalent hydroperoxides per kg of oil (mmol eq HP kg⁻¹ oil) with 27000 M⁻¹ cm⁻¹ as the molar extinction coefficient of CD at 233 nm.

### Formation of Total Aldehydes.

The para-anisidine value (p-AV), a measure of total aldehydes, was used to assess the formation of secondary lipid oxidation products (AOCS, 1998). In short, 1 mL saturated sodium chloride solution and 5 mL hexane/isopropanol (1/1, v/v) were added per aliquot of incubated emulsion (2.1 mL). Mixtures were vortexed followed by centrifugation at 2000×g for 8 minutes at 4° C. The upper hexane layer (>2 mL) was collected and placed on ice for 3 minutes, followed by centrifugation at 20238×g for 1 minute. The absorbance of the supernatant was measured at 350 nm with pure hexane as a blank (Ab). In a centrifugation vial, 1 mL of the supernatant was mixed with 0.2 mL 2.5 g/L para-anisidine in acetic acid solution. After exactly 10 min, the absorbance was measured at 350 nm using 1 mL pure hexane mixed with 0.2 mL 2.5 g/L para-anisidine in acetic acid solution, incubated for 10 min, as a blank (As). The para-anisidine value (pAV, arbitrary units) was calculated as follows:

$$pAV = \frac{( 1.2 As - Ab )}{m}$$

Where m is the concentration of oil (g/mL).

### Example 1. Oxidative Stability of a Conventional Sodium Caseinate-Stabilized Oil in Water Emulsion Containing Tripalmitin Compared to an Emulsion of the Invention Comprising Tripalmitin Colloidal Particles Prepared as Detailed in the Material and Methods Section

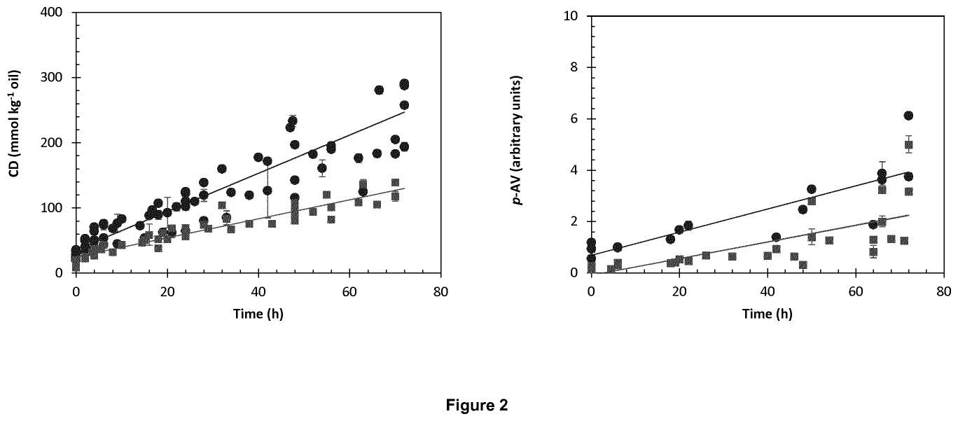

The oxidative stability of a Pickering emulsion stabilized by tripalmitin colloidal particles (PTP) has been evaluated by both conjugated dienes (primary oxidation products) and p-anisidine (secondary oxidation products) in comparison to a conventional sodium caseinate-stabilized emulsion containing HMP fat in the same amount (FIG. 1). Lipid oxidation in both emulsions was accelerated by 200 μM FeSO₄/EDTA. The data obtained showed that tripalmitin colloidal particles exert a protective effect on the corresponding Pickering emulsion as both CD-LOOH and p-AV raised more slowly compared to the same emulsion (conventional) wherein tripalmitin is dissolved in the interior of the oil droplet (FIG. 2).

### Example 2. Oxidative Stability of a Conventional Sodium Caseinate-Stabilized Oil in Water Emulsion Containing Palm Stearin Compared to an Emulsion of the Invention Comprising Palm Stearin Colloidal Particles Prepared as Detailed in the Material and Methods Section

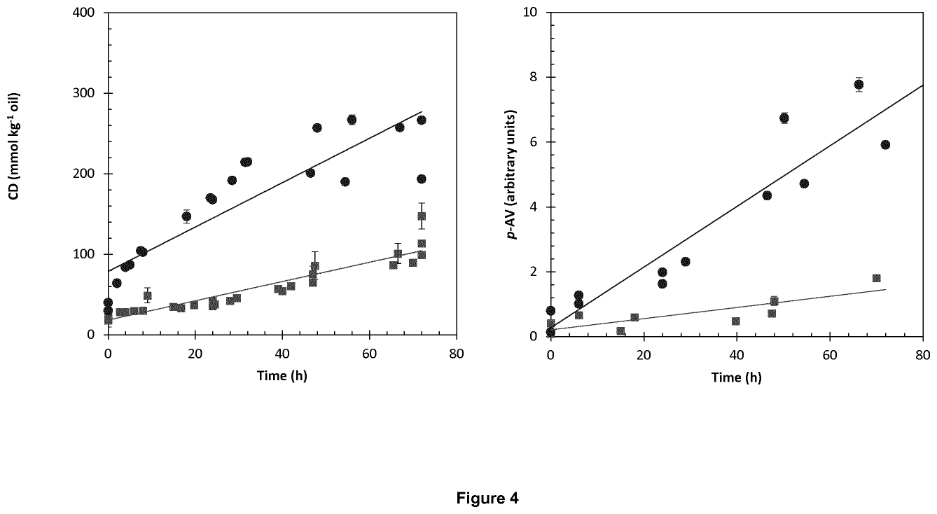

The same experiment as in Example 1 was repeated using palm stearin instead of tripalmitin. A Pickering emulsion stabilized by colloidal particles formed by palm stearin (PPS) was evaluated in comparison to a conventional sodium caseinate-stabilized emulsion containing HMP fat in the same amount (FIG. 3). Lipid oxidation in both emulsions was accelerated by 200 μM FeSO₄/EDTA. The effect previously seen with tripalmitin was exacerbated with palm stearin. The colloidal particles in this example exerted a huge antioxidative effect on the corresponding Pickering emulsion. This has been demonstrated on both primary (conjugated dienes) and secondary (p-anisidine) oxidation products (FIG. 4).

### Example 3. Characterization of the Particle Size Distribution of Particles Prepared from One or More Biological Materials in Suspension where the Biological Material is Obtained from a Photosynthetic Organism

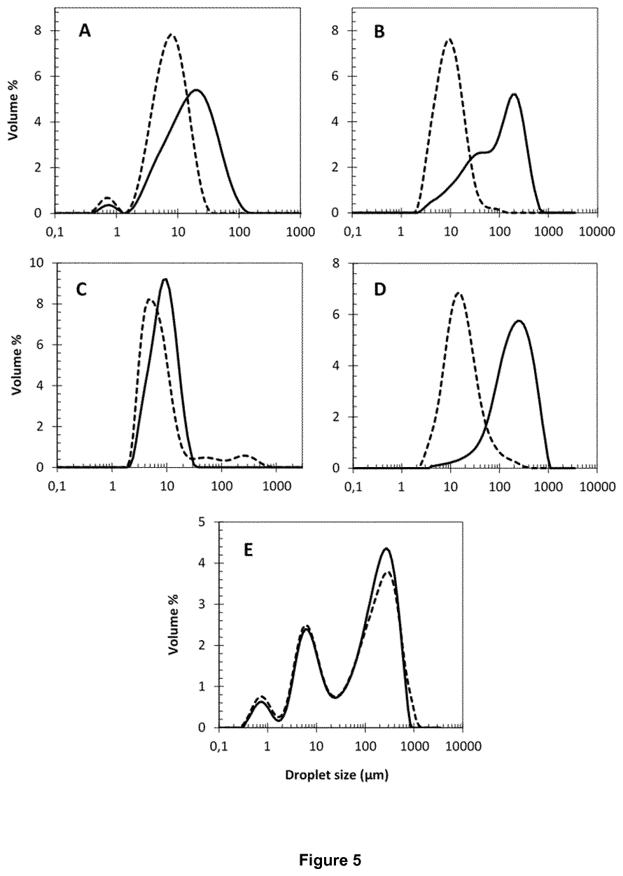

The characterization of the particle size distribution of some representative natural powders suspended in water at 1% (w/w) was performed using static light scattering (Malvern Mastersizer 3000, Malvern Instruments Ltd., Malvern, Worcestershire, UK) with a refractive index particle of 1.45 and an adsorption index of 0.01 (FIG. 5). Both micronized and non-micronized powders (matcha tea raw material, spinach leaves raw material, spirulina extraction cake, pineapple fibers, and rosemary leave extraction cake) were tested.

Interestingly, no particle size below 0.2 μm was measured demonstrating that the particles used were not nanoscale.

Particles from pineapple and spinach leave powders were found to possess a higher particle size than matcha tea and spirulina cake. The non-micronized spinach leaves particles contained particles of various diameters (i.e. a polydisperse distribution) with a main peak at 200 μm, whereas the micronized particles of the same material contained particles of uniform size (i.e. had a monodisperse distribution) with an average particle size of 8 μM. These results also show that micronization not only has a significant impact on the reduction of particle size, but also on the size distribution.

Matcha tea powder and spinach leave particles were polydispersed before micronization, but monodispersed after processing. Spirulina cake became more polydispersed once micronized, whereas pineapple kept a monodisperse distribution. The rosemary cake powder in both micronized and non-micronized form was polydisperse in size, appearing as big and small particles. Nevertheless, unlike the other materials, the particle size distribution was not significantly affected by micronization.

### Example 4. Chemical Characterization of Particles Prepared from One or More Biological Materials where the Biological Material is Obtained from a Photosynthetic Organism

A chemical characterization of a representative set of micronized or non-micronized particles has been done and is presented in Tables 1 and 2.

TABLE 1
Composition table of some representative particles. Polyphenol content values with asterisks
were determined by HPLC, while the others were from the Folin-Ciocalteu method.

| | | Free | Total | Free | Total | | | |
|---|---|---|---|---|---|---|---|---|
| Samples | Maltodextrins | sugars | Sugars | glucose | glucose | Starch | Ash | Polyphenols |
| Micronized | ND | ND | 0.17 | ND | ND | ND | 1.36 | 92.91* |
| curcumin | | | | | | | | |
| Non-micronized | ND | ND | 0.19 | ND | ND | ND | ND | 92.75* |
| curcumin | | | | | | | | |
| Micronized | ND | 1.62 | 4.89 | ND | ND | ND | 1.50 | 11.27 |
| rosemary | | | | | | | | |
| cake | | | | | | | | |
| Non-micronized | ND | 2.2 | 4.17 | 0.25 | ND | ND | 1.58 | 10.95 |
| rosemary | | | | | | | | |
| Micronized | 42.36 | ND | 84.61 | ND | 78.23 | 27.26 | ND | 1.33 |
| red radish | | | | | | | | |
| Non-micronized | 44.06 | 0.52 | 94.01 | 0.52 | 77.1 | 24.10 | ND | 1.62 |
| red radish | | | | | | | | |
| Micronized | ND | 0.2 | 5.57 | 0.12 | ND | ND | 17.70 | 0.15 |
| spirulina | | | | | | | | |
| Non-micronized | ND | 0.2 | 5.79 | 0.2 | ND | ND | 17.79 | 0.36 |
| spirulina | | | | | | | | |
| Micronized | ND | 3.38 | 16.61 | 0.15 | ND | ND | 4.72 | 14.54 |
| matcha tea | | | | | | | | |
| Non-micronized | ND | 4.95 | 15.7 | 0.73 | ND | ND | 4.70 | 21 |
| matcha tea | | | | | | | | |
| Micronized | ND | 1.16 | 33.0 | 0.52 | ND | ND | 12 | 0.47 |
| pineapple | | | | | | | | |
| Non-micronized | ND | 0.88 | 31.06 | ND | ND | ND | 1.09 | 1.45 |
| pineapple | | | | | | | | |
| Micronized | ND | 4.82 | 17.21 | 1.16 | 4.95 | 3.38 | 14.19 | 1.17 |
| spinach | | | | | | | | |
| Non-micronized | ND | 5.22 | 17.75 | 0.79 | 5.14 | 3.87 | 15 | 1.11 |
| spinach | | | | | | | | |

TABLE 2
Follow-up composition table of some representative particles.

| | | | | Neutral | | | |
|---|---|---|---|---|---|---|---|
| | | Total | | detergent | | | |
| Samples | Proteins | nitrogen | Cellulose | Fibres | Fibres | Lignin | Hemicellulose |
| Micronized | <0.08 | <0.5 | <2.00 | 2.20 | <0.50 | <0.50 | 2.20 |
| curcumin | | | | | | | |
| Non-micronized | 1.2 | 0.18 | 14.6 | 34.2 | 17.7 | 16.8 | 16.5 |
| rosemary | | | | | | | |
| Micronized red | 0.50 | 0.08 | <2.00 | 3.40 | 1.20 | <0.50 | 2.20 |
| radish | | | | | | | |
| Non-micronized | 0.90 | 0.14 | <2.00 | 3.80 | 1.30 | <0.50 | 2.50 |
| red radish | | | | | | | |
| Micronized | 57.8 | 9.25 | <2.00 | 5.50 | 3.30 | <0.50 | 2.20 |
| spirulina | | | | | | | |
| Non-micronized | 58.10 | 9.30 | <2.00 | 1.20 | 0.60 | <0.50 | <0.60 |
| spirulina | | | | | | | |
| Micronized | 22.70 | 3.64 | 2.80 | 5.10 | 2.90 | 1.20 | 2.20 |
| matcha | | | | | | | |
| Non-micronized | 22.00 | 3.52 | 4.60 | 27.60 | 16.40 | 8.50 | 11.20 |
| matcha | | | | | | | |
| Micronized | 1.80 | 0.28 | 17.90 | 26.20 | 13.20 | 7.10 | 13.00 |
| pineapple | | | | | | | |
| Non-micronized | 1.60 | 0.26 | 28.60 | 76.20 | 35.60 | 9.60 | 40.60 |
| pineapple | | | | | | | |
| Micronized | 28.85 | 4.62 | 6.95 | 15.6 | 5.45 | 2.75 | 10.05 |
| spinach | | | | | | | |
| not micronized | 28.00 | 4.48 | 6.70 | 15.10 | 8.40 | 1.30 | 6.70 |
| Spinach | | | | | | | |

### Example 5. Morphological Characterization of Particles Prepared from One or More Biological Materials where the Biological Material is Obtained from a Photosynthetic Organism in their Dry Form

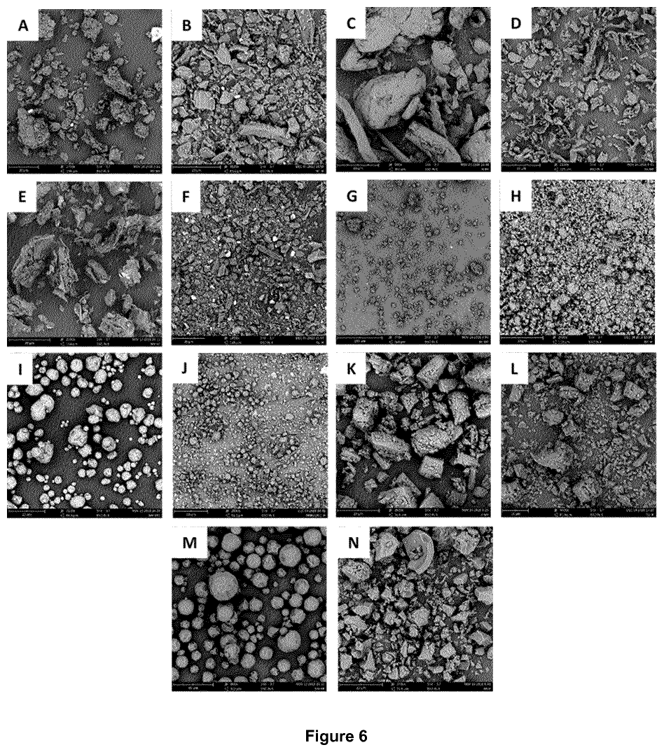

The particles dried form microstructure was accessed using scanning electron microscopy (SEM). The non-micronized curcuma particles had a polyhedral shape whereas the micronized sample had an irregular shape (FIG. 6). The non-micronized red radish and spirulina cake particles had initially a spherical shape, but after processing, both micronized samples were irregular. Therefore, the results showed that for these particles, the microstructure was broken down by ultrasonification (i.e. micronization).

For matcha tea powder, pineapple, rosemary cake and spinach leaves particles shape did not seem to be affected by micronization as both non-micronized and micronized particles presented an irregular structure before and after processing. Moreover, the matcha tea powder, spinach leaves and rosemary cake particles presented high porosity, whereas the pineapple particles did not.

### Example 6. Characterization of the Physical Stability of the Pickering Oil-in-Water Emulsions of the Invention Stabilized by Particles Prepared from One or More Biological Materials where the Biological Material is Obtained from a Photosynthetic Organism

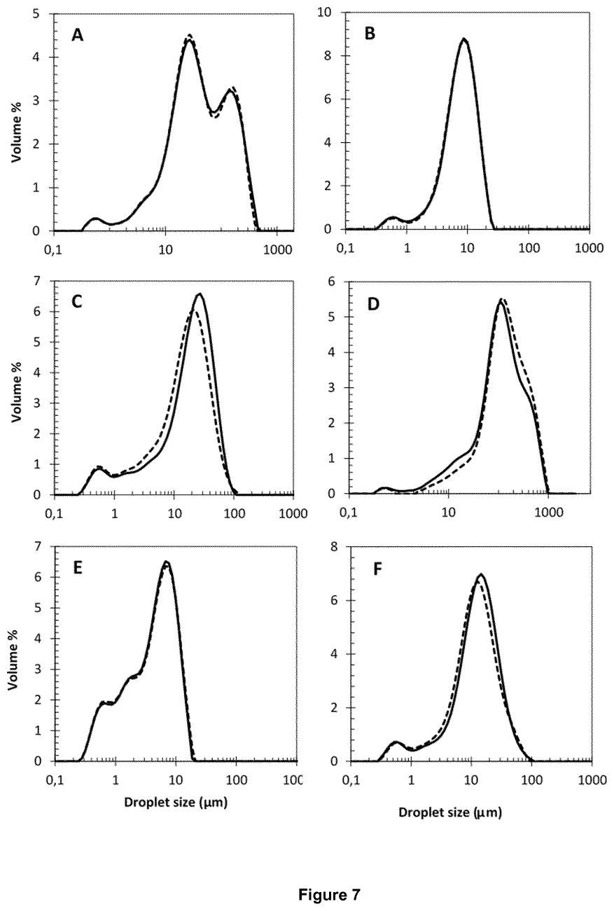

In this example, the emulsion forming and stabilizing ability of the natural particles of the invention was assessed through the particle size distribution of the corresponding oil-in-water emulsions. FIG. 7 shows that spinach leaves (A), spirulina cake (B), matcha tea (C), and pineapple fibers (D), all in their non-micronized form were able, when added at 5% w/w, to form and stabilize Pickering oil-in-water emulsions over three months at 4° C.

These emulsions were formed and stabilized in an emulsifier-free medium and were compared to two conventional oil-in-water emulsions stabilized by Tween 60 at 1% (w/w) (FIG. 7E) or egg yolk at 5% (w/w) (FIG. 7F). All emulsions were prepared in a 50 mM acetate buffer pH 4.5 and contained 0.1 wt % potassium sorbate as antimicrobial.

### Example 7. Characterization of the Physical Stability of the Pickering Oil-in-Water Emulsions of the Invention Stabilized by Particles Prepared from One or More Biological Materials where the Biological Material is Obtained from a Photosynthetic Organism when NaCl is Added

In this example, the emulsion forming and stabilizing ability of particles prepared from one or more biological materials where the biological material is obtained from a photosynthetic organism were assessed in the presence of NaCl through the particle size distribution of the corresponding oil-in-water emulsions.

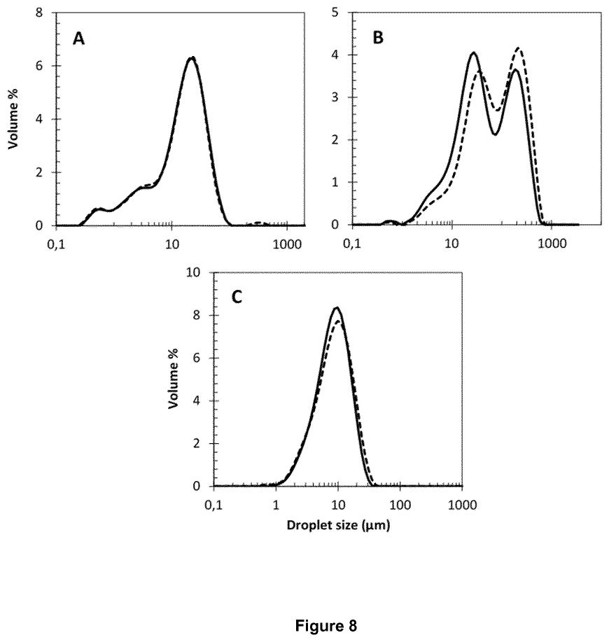

FIG. 8 shows that matcha tea (A), spinach leaves (B), and spirulina cakes (C), all in their non-micronized form, were able, when added at 5% w/w, to form and stabilize (during one month at 4° C.) Pickering oil-in-water emulsions prepared in a 50 mM acetate buffer pH 4.5 in presence of a substantial level of salt.

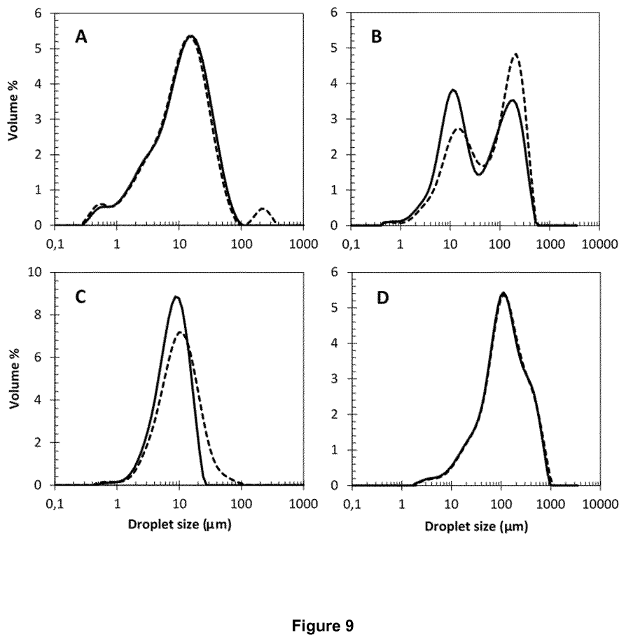

FIG. 9 shows similar results for non-micronized matcha tea (A), spinach leaves (B), spirulina cakes (C), and pineapple fibers (D) when the exact same emulsions were prepared in a 50 mM phosphate buffer pH 7.0 instead of an acetate buffer.

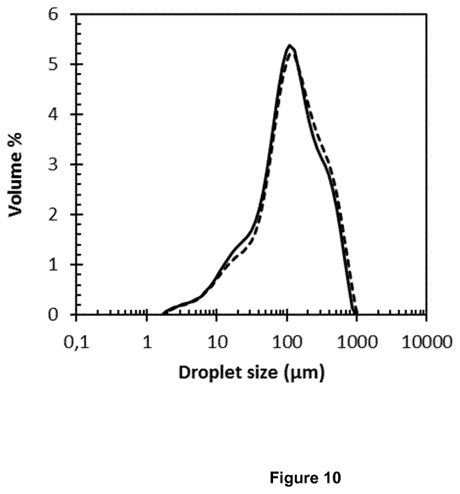

Finally, FIG. 10, shows similar results for non-micronized pineapple fibers when the exact same emulsion was prepared in unbuffered ultrapure water of “Type 1” as defined by ISO3696 (for example, milliQ water).

This series of data clearly indicates that the Pickering oil-in-water emulsions of the invention can be formed and stabilized for a significant amount of time in presence of salt which is known for having in some cases disturbing effect on the physical stability of oil-in-water emulsions.

### Example 8. Characterization of the Physical Stability of the Pickering Oil-in-Water Emulsions of the Invention Stabilized by Particles Prepared from One or More Biological Materials where the Biological Material is Obtained from a Photosynthetic Organism at Neutral and Acidic pH

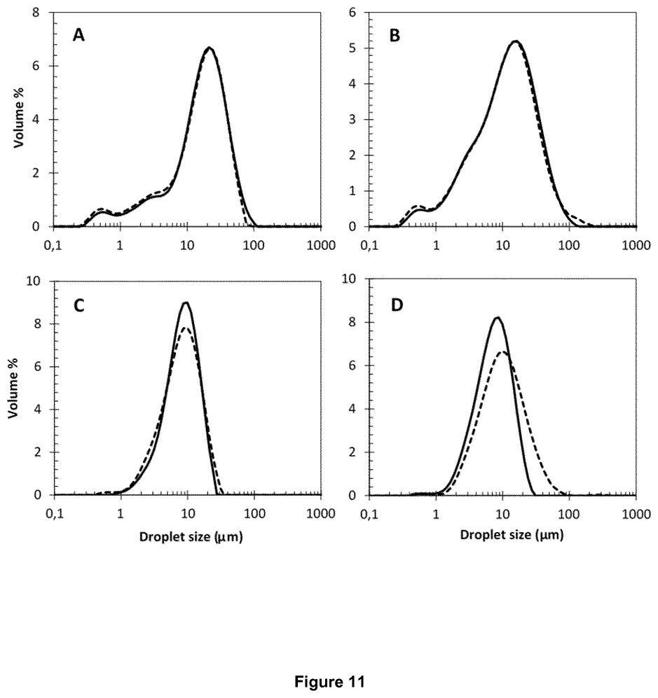

Here, the emulsion forming and stabilizing ability of particles prepared from one or more biological materials where the biological material is obtained from a photosynthetic organism was assessed at different pH through the particle size distribution of the corresponding oil-in-water emulsions. FIG. 11 shows that matcha tea powder at pH 4.5 (A) and pH 7.0 (B), as well as spirulina cake powder at pH 4.5 (C) and pH 7.0 (D), all in their non-micronized form, were able, when added at 5% w/w, to form and stabilize (during one month at 4° C.) Pickering oil-in-water emulsions in presence of a substantial level of salt. Emulsions at pH 4.5 were prepared in a 50 mM acetate buffer, while those at pH 7.0 were in a 50 mM phosphate buffer.

### Example 9. Oxidative Stability of a Conventional Tween 60-Stabilized Oil in Water Emulsion Compared to a Pickering Oil-in-Water Emulsion of the Invention Stabilized by 5% (w/w) of a Non-Micronized Spirulina Cake Powder

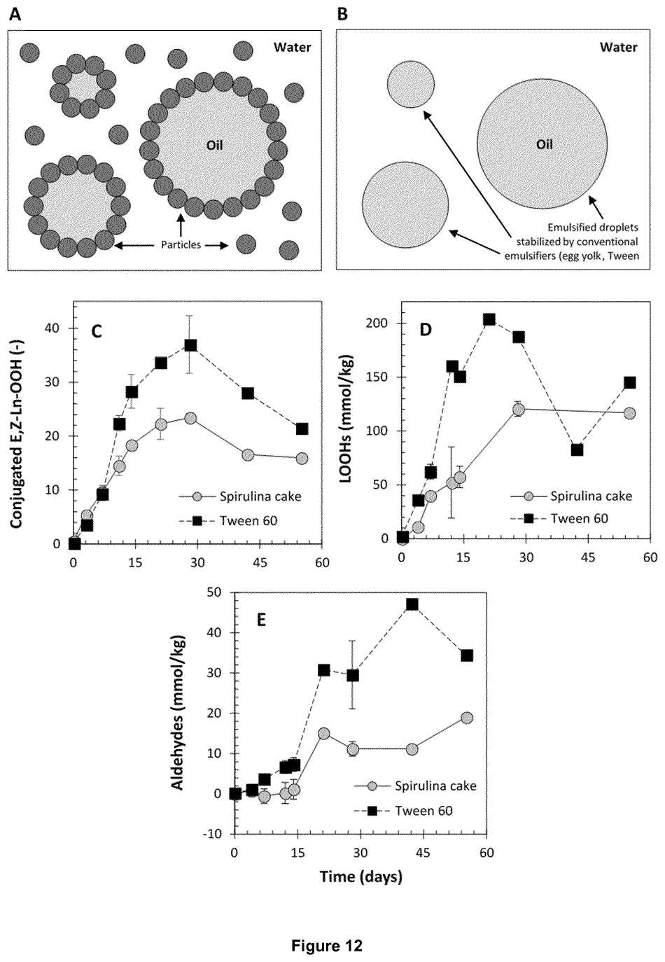

The oxidative stability of a conventional Tween 60-stabilized oil in water emulsion has been evaluated through the level of conjugated dienes (conjugated E,Z-Ln-OOH, primary oxidation products), lipid hydroperoxides (LOOHs, primary oxidation products) and aldehydes (secondary oxidation products) in comparison to a Pickering oil-in-water emulsion (emulsion of the invention) stabilized by 5% (w/w) of a non-micronized spirulina cake powder (FIG. 12). Lipid oxidation in both emulsions was natural (i.e. non-accelerated by oxidation catalyst(s) other than those naturally present in the systems). After four months of incubation at 25° C., data shows that spirulina cake powder exerts a surprising protective effect on the corresponding Pickering oil-in-water emulsion as all oxidation markers raised more slowly compared to the conventional emulsion stabilized by Tween 60 (a standard emulsifier used in industry). Thus, the particles of spirulina cakes act as natural antioxidant colloids or particles.

### Example 10. Oxidative Stability of a Conventional Egg Yolk-Stabilized Oil in Water Emulsion Compared to Two Pickering Oil-in-Water Emulsions of the Invention Stabilized by 5% (w/w) of a Non-Micronized Matcha Tea Powder or 5% (w/w) of a Non-Micronized Spinach Leave

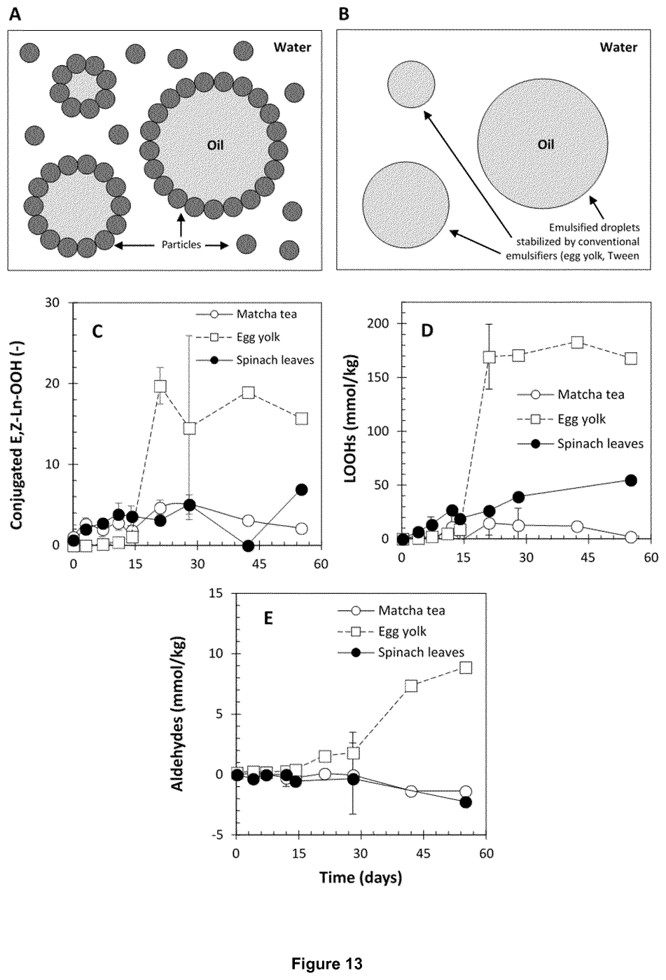

The oxidative stability of a conventional egg yolk-stabilized oil in water emulsion has been evaluated through the level of conjugated dienes (conjugated E,Z-Ln-OOH, primary oxidation products), lipid hydroperoxides (LOOHs, primary oxidation products) and aldehydes (secondary oxidation products) in comparison to two Pickering oil-in-water emulsions (emulsions of the invention) stabilized by 5% (w/w) of non-micronized matcha tea powder or non-micronized spinach leaves (FIG. 13). Lipid oxidation in both emulsions was natural (i.e. non-accelerated by oxidation catalyst(s) other than those naturally present in the systems). After four months of incubation at 25° C., data shows that both natural powders exert a surprising protective effect on the corresponding Pickering oil-in-water emulsions as all oxidation markers raised more slowly compared to the conventional emulsion stabilized by egg yolk (a standard emulsifier used in industry). Thus, the particles of matcha tea and those of spinach leaves act as natural antioxidant colloids or particles.

### Example 11. Characterization of the Ability of Particles Prepared from One or More Biological Materials where the Biological Material is Obtained from a Photosynthetic Organism to Form and Stabilize Water-in-Oil Emulsions (the Emulsions of the Invention)

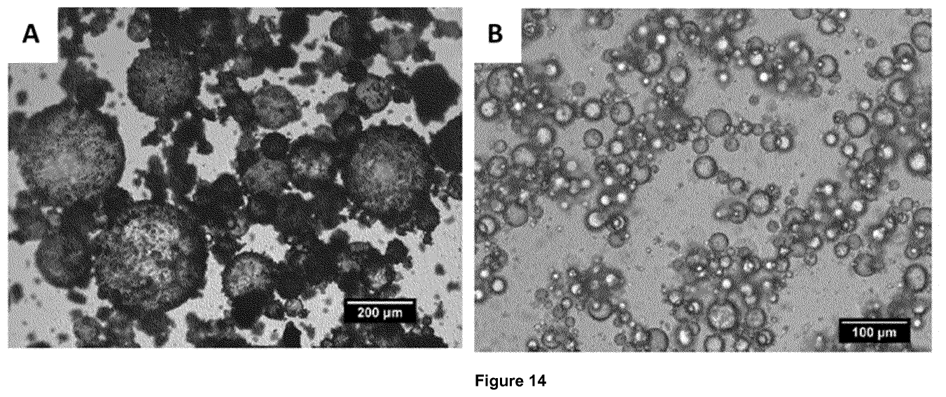

Unexpectedly, we were able to form 10% water-in-oil emulsions (reverse emulsions) using 1% (w/w) non-micronized curcuma extract particles (FIG. 14A) or 2.5% (w/w) non-micronized rosemary leave extraction cake particles (FIG. 14B)

### Example 12. Characterization of the Ability of Particles Prepared from One or More Biological Materials where the Biological Material is Obtained from a Photosynthetic Organism to Form and Stabilize Water-in-Oil Emulsions (the Emulsions of the Invention) after that the Particles have been Washed with Water

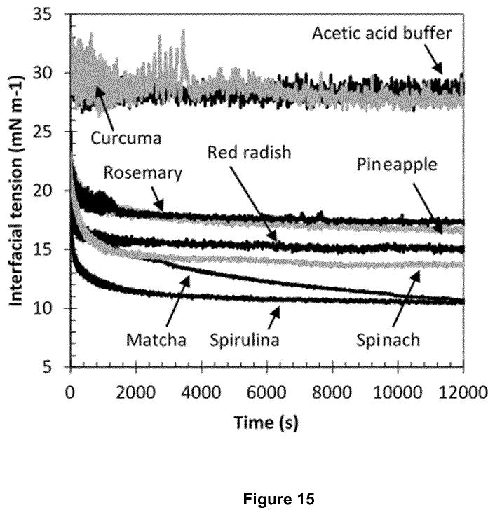

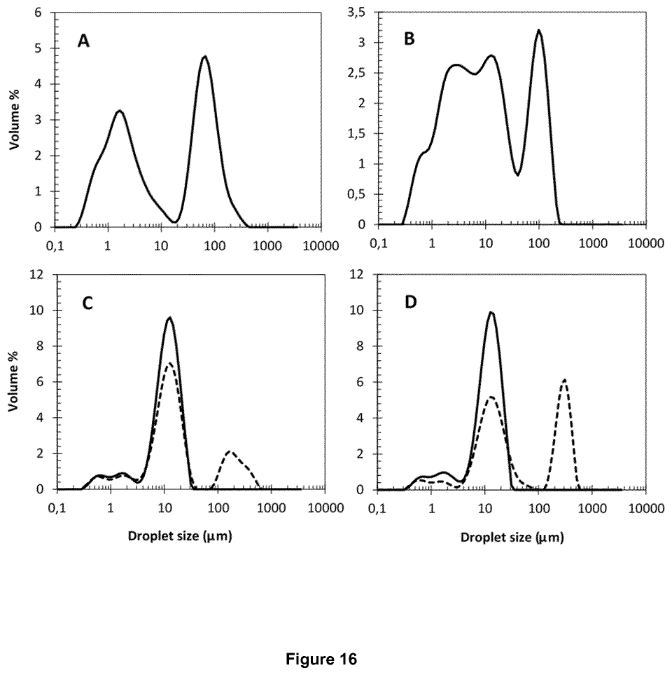

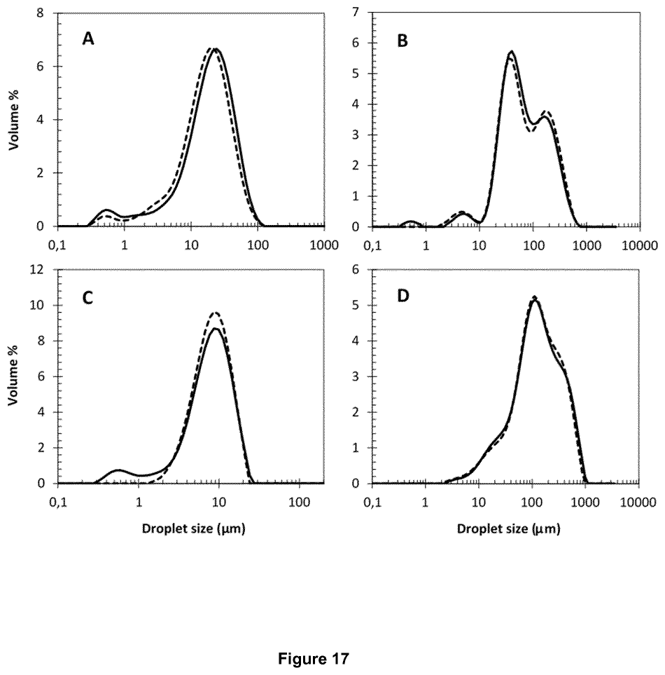

When washing the natural particles with water, we have unexpectedly found that the resulting supernatants exert, for most of them, a significant, although relatively modest, tensio-activity (i.e. the ability to decrease the tension at the interface between stripped sunflower oil and water (FIG. 15)). Hence, to decipher if the stabilizing effect previously seen in Examples 6, 7, and 8 was merely due to the tensio-activity of some surface-active molecules contained in the powders and removable by washing or was more specifically due to a Pickering (mechanical) stabilization mechanism, we recapitulated some physical stability tests on Pickering emulsions stabilized by the washed particles. Interestingly, the washed particles were all able to physically stabilize the resulting oil-in-water emulsions (FIG. 17). We also tested the supernatant resulting from the washing procedure and found that they were not able to stabilize oil-in-water emulsions (FIG. 16), thus clearly showing that the stabilizing effect is conveyed by a true Pickering mechanism and not by a conventional emulsifying effect.

### Example 13. Comparison of Sunflower Oil-in-Water Emulsions where the Antioxidant (α-Tocopherol) is Either (i) in the Palmitin Colloidal Particles (the Emulsion of the Invention Prepared as Detailed in the Materials and Methods Section) or (ii) within the Interior of the Oil Droplets

Two Pickering emulsions prepared as detailed above were prepared. One with a-tocopherol incorporated in the colloidal particles (FIG. 18a) and one with α-tocopherol in the liquid sunflower oil droplets (FIG. 18b).

Oxidation was accelerated with 200 μM FeSO₄/EDTA at 25° C. The emulsions were then tested for both primary oxidation products such as conjugated dienes and secondary oxidation products such as p-anisidine aldehydes. As shown in FIGS. 18c and 18d, less oxidation occurred in the emulsion when α-tocopherol was incorporated in the colloidal particles compared to the same emulsion where α-tocopherol was solubilized in the interior of the oil droplets.

The stability of α-tocopherol in each emulsion was then investigated by extracting a-tocopherol from either the colloidal particles or emulsion droplets as described in FIGS. 18a and b.

HPLC analysis of the α-tocopherol showed that incorporating α-tocopherol into the colloidal particles provided significant protection to the antioxidant (FIG. 18e).

This unexpected effect brings an additional advantage to the formulation studied here since α-tocopherol in particular, and phenol-bearing compounds in general, are very sensitive to oxidation mediated by lipid oxidation products such as free radicals.

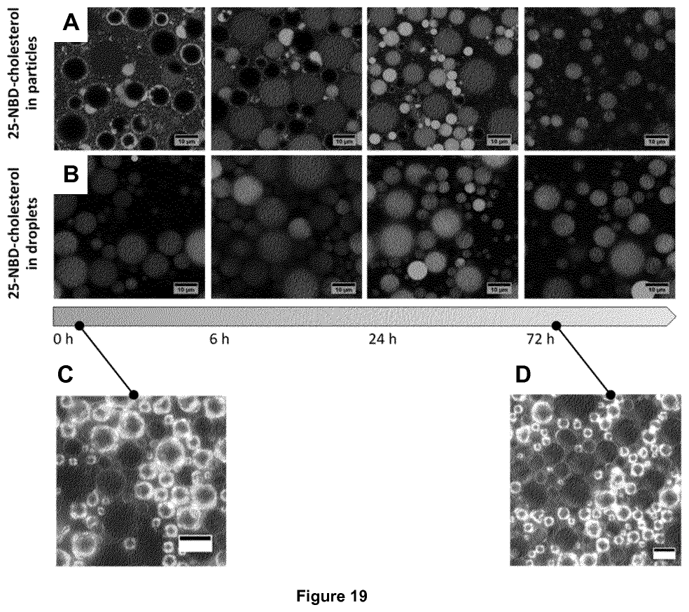

To further characterize the improvement in antioxidant activity of α-tocopherol when formulated in colloidal particles (FIGS. 18c and d) along with the protection effect of such a formulation on tocopherol itself (FIG. 18e), confocal laser scanning microscopy at different time intervals (0, 6, 24, and 72 hours) was used to image the emulsion labelled with a fluorescent analog of α-tocopherol (25-NBD-cholesterol) located in the colloid particles (FIG. 19A).

As it can be seen in FIG. 19A, when the fluorescent dye is in the colloidal particles attached at the interface (i.e. Pickering particles), a strong green fluorescence is present around all lipid droplets at 0 min, forming a ring-like pattern. This shows that a significant part of the α-tocopherol fluorescent analog is located at the interface within adsorbed colloidal particles. With time, α-tocopherol is only slowly released from the colloidal particles to the liquid droplets. This can be seen through the decrease of the black-to-green droplet ratio from 0 to 72 hours.

In contrast, when the fluorescent analog of α-tocopherol is in the liquid oil droplet (FIG. 19B), all droplets are green, demonstrating that the fluorescent analog of α-tocopherol immediately reaches a dynamical equilibrium within all oil droplets.

This shows that incorporation of the anti-oxidant (i.e. α-tocopherol) within the colloidal particles allows the anti-oxidant (i.e. α-tocopherol) to locate at the interface and provide an improved anti-oxidant effect whilst being protected by the colloidal particles, and that the anti-oxidant (i.e. α-tocopherol) is only slowly released from the colloidal particles, thus maintaining the anti-oxidant effect.

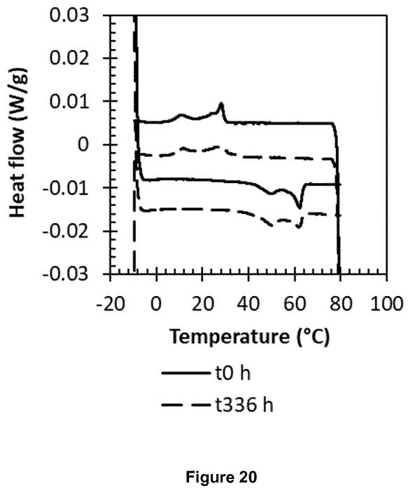

Finally, polarized light microscopy (FIGS. 19C and 19D) and differential scanning calorimetry (DSC) analyses (FIG. 20) showed that the colloidal particles adsorbed at the oil-water interface, remained physically intact over the timescale of the experiment. This result suggests that the diffusion of α-tocopherol fluorescent analog (hence, by analogy, the diffusion of α-tocopherol) is caused by the solubilization of the colloidal particles in the liquid oil phase over time, which is in line with the high long-term physical stability of those emulsions.

### Example 14. Comparison of Sunflower Oil-in-Water Emulsions where the Antioxidant (Carnosic Acid) is Either (i) in the Palmitin Colloidal Particles (the Emulsion of the Invention Prepared as Detailed in the Materials and Methods Section) or (ii) within the Interior of the Oil Droplets

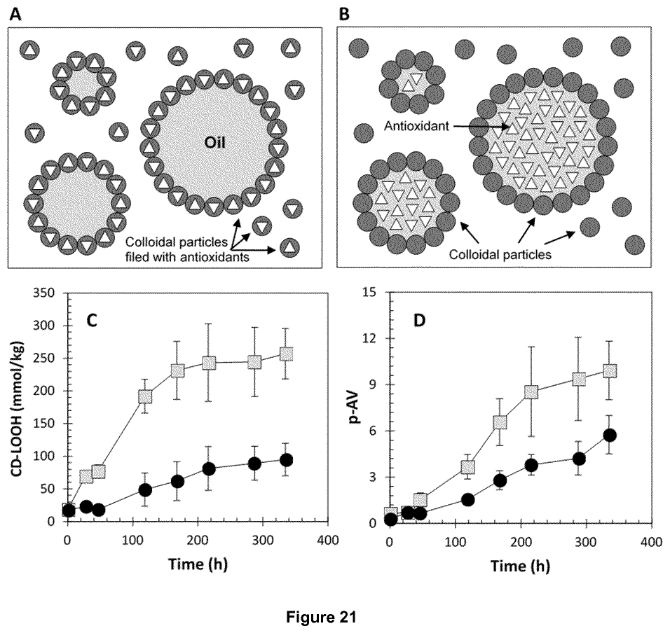

The exact same experimental design as Example 13 was reproduced except carnosic acid was used to replace α-tocopherol. The data presented in FIG. 21 clearly showed that the same antioxidant effect is obtained with this diterpene phenolic antioxidant, indicating that the colloidal particles of the invention can serve as interfacial reservoirs for many antioxidant molecules to provide an antioxidant-enhancing effect.

### Example 15. Comparison of Sunflower Oil-in-Water Emulsions where the Antioxidant (α-Tocopherol) is Either (i) in Palmitin Colloidal Particles not Adsorbed at the Interface or (ii) is within the Interior of the Oil Droplets

To investigate whether the improvement of the antioxidant activity of α-tocopherol formulated in colloidal particles was specifically due to the interfacial anchorage of these particles, two types of conventional sodium caseinate-stabilized stripped sunflower oil-in-water emulsions comprising an aqueous suspension of colloidal particles were prepared.

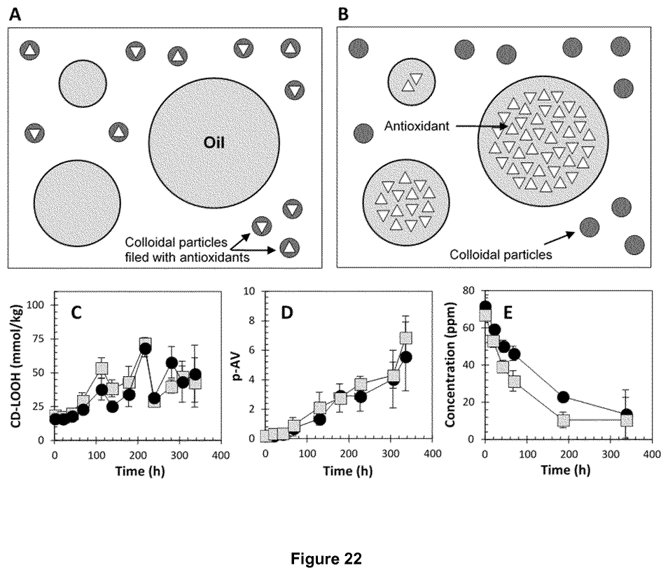

The emulsion and suspension compositions were identical, but the antioxidant was located either in the suspended colloidal particles (FIG. 22a) or in the core of the oil droplets (FIG. 22b). The main difference compared to Example 3 was that the colloidal particles were not adsorbed to the interface and were instead added as a particle suspension in the aqueous phase (i.e. unadsorbed).

Oxidation was accelerated with 200 μM FeSO₄/EDTA at 25° C. The emulsions were then tested for both primary oxidation products such as conjugated dienes and secondary oxidation products such as p-anisidine aldehydes. As shown in FIGS. 22c and 22d, the oxidative stability of both emulsions was quite similar, and both emulsions contained a higher concentration of oxidation products than the emulsion of Example 3, an emulsion of the invention where the anti-oxidant is contained within the colloidal particles attached to the oil/water interface.

Extraction of α-tocopherol from the unadsorbed colloidal particles or emulsion droplets, followed by HPLC showed the protective effect conferred by the colloidal particles in Example 13 (FIG. 18e) was dramatically reduced when the particles were not absorbed at the droplet surface (FIG. 22e).

Again, this shows that the emulsion of the invention (Examples 13 and 14) provides a much better protection to the antioxidant compounds than the two described emulsions of Examples 15. The attachment of the antioxidant-loaded particles to the interface (i.e. true Pickering particles) is thus required to have a beneficial effect in terms of antioxidant activity.

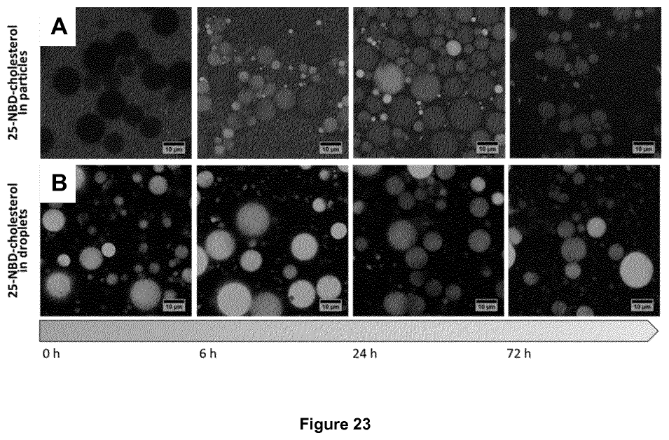

Finally, laser scanning microscopy at different time intervals (0, 6, 24, and 72 hours) was used to image the emulsion labelled with a fluorescent analog of α-tocopherol (25-NBD-cholesterol) located in the un-adsorbed colloid particles (FIG. 23A) or directly in the oil droplets (FIG. 23B).

As it can be seen, when the fluorescent dye is in the suspended particles, a green fluorescence is homogenously distributed in the external aqueous phase at 0 min. At that incubation time, no green droplets can be observed. This shows that at 0 hour, no colloidal particles are adsorbed at the oil/water interface. Instead they are suspended in the aqueous phase as above-mentioned where they are quite inefficient to counteract lipid oxidation. With time, a depletion of the green background is paralleled by an increase of the green droplet, clearly showing that the fluorescent analog of α-tocopherol progressively diffuse from the aqueous phase to the oil droplet interior.

At 72 hours, the black-to-green droplet ratio is similar (FIG. 22A), if not identical, as the one depicted in FIG. 23B where the dye was initially located in the oil droplet core.

### Example 16. Comparison of Pickering Sunflower Oil-in-Water Emulsions where the Antioxidant (α-Tocopherol) is Either (i) in Palmitin Colloidal Particles not Adsorbed at the Interface and Refrained to Diffuse in the Oil Interior by a “Pickering Barrier” or (ii) is within the Interior of the Oil Droplets

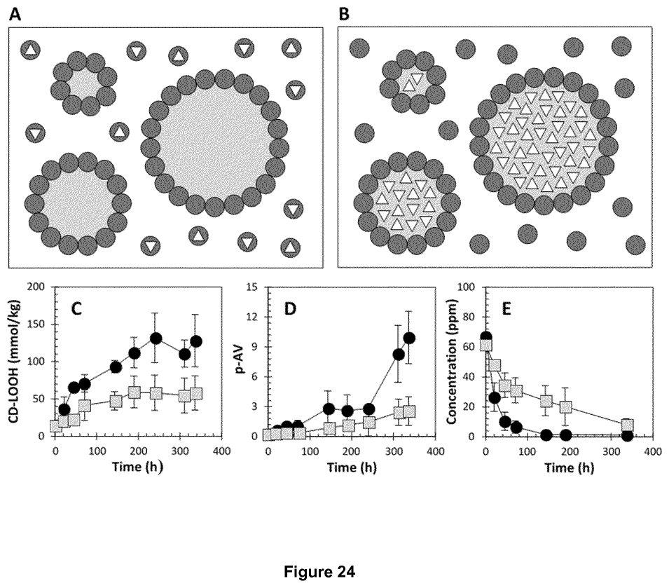

To decipher the respective contribution of each population of colloidal particles (adsorbed vs. non-adsorbed at the interface) in the enhancing effect on α-tocopherol antioxidant activity seen in Example 13 (the emulsion of the invention), a Pickering emulsion stabilized by α-tocopherol-free colloidal particles attached to the interface was prepared, to which a-tocopherol-loaded colloidal particles were added post homogenization, resulting in the a-tocopherol-loaded colloidal particles not attaching to the interface (FIG. 24A).

This emulsion was compared to the same Pickering oil-in-water emulsion except the a-tocopherol was located in the core of the oil droplets (FIG. 24B).

This comparison allowed the assessment of the role of antioxidant-loaded CLPs in the continuous phase, while keeping the interfacial structure similar to that of the emulsion of the invention (Example 13).

Lipid oxidation proceeded significantly faster in Pickering emulsions containing a-tocopherol exclusively in the colloidal lipid particles (FIGS. 24C and 24D). Thus, we can conclude that the antioxidant-loaded particles suspended in the aqueous phase of the emulsion of the invention (Example 13) have no contribution to the antioxidant activity-improving effect. Indeed, here it is clearly shown that this precise antioxidant-loaded particle subpopulation is less effective at inhibiting lipid oxidation than α-tocopherol directly formulated in the oil phase.

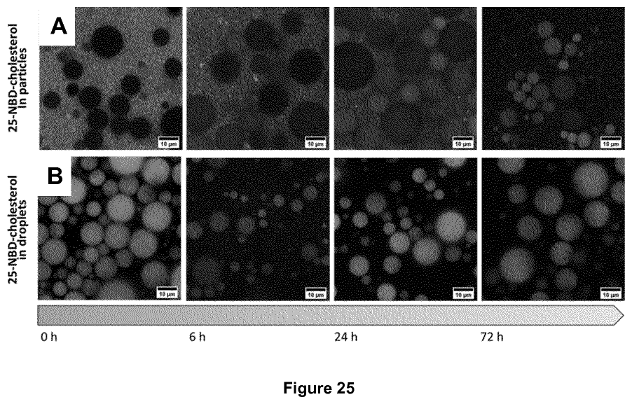

As in previous Examples, emulsions with similar construction principle were also prepared with 25-NBD cholesterol. The diffusion of the fluorescent probe from the colloidal particles present in the aqueous phase to the emulsion droplet core during incubation was much slower compared to the protein-stabilized emulsion (FIG. 25A), which confirms the beneficial effect that the colloidal particles provide to the emulsions of the invention as seen in Example 13.

### Example 17. Comparison of Pickering Sunflower Oil-in-Water Emulsions with Different Concentrations of an Antioxidant (α-Tocopherol) which is Located Either (i) in the Tripalmitin Colloidal Particles (the Emulsion of the Invention Prepared as Detailed in the Material and Methods Section) or (ii) within the Interior of the Oil Droplets

To investigate to what extent our hierarchical emulsion design presented in Example 13 boosted the antioxidant efficiency of an antioxidant as compared to a control emulsion where the antioxidant is located within the interior of the oil droplets, an emulsion of the invention was produced with a reduced α-tocopherol content from 90 ppm to 45, then 22.5 ppm (2 to 4 times lower).

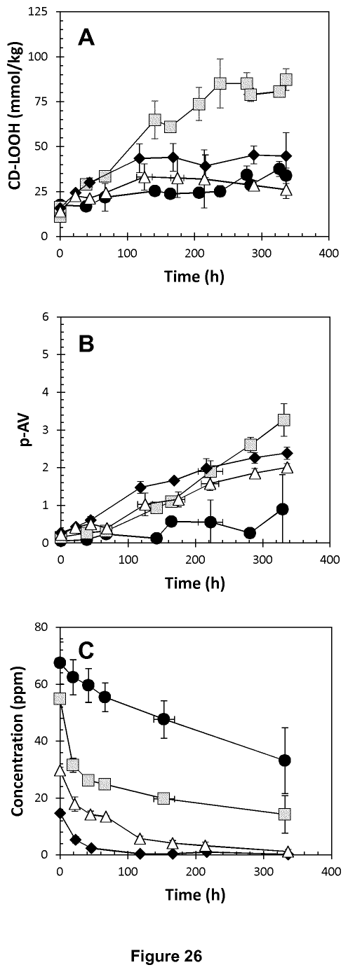

Interestingly, we found that, when formulated in the emulsion of the invention, tocopherol can be drastically reduced (at least 2 to 3 times) and still provide a protection against lipid oxidation which is superior or equal to that obtained with 2 to 3 times higher concentrations of α-tocopherol in the control emulsion where the antioxidant is formulated in the interior of the lipid droplets (FIG. 26).

### Example 18. Physical and Morphological Characterization of Tripalmitin Colloidal Particles with or without Antioxidant (α-Tocopherol)

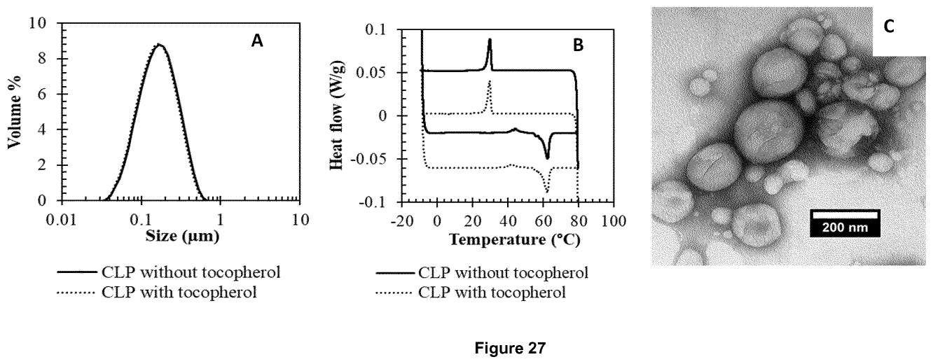

In this example, we characterized the particles containing or not α-tocopherol using particle size distribution (A), differential scanning calorimetry (A) and TEM (A) (FIGS. 27A, B and C).

### Example 19. Physical and Morphological Characterization of Pickering Sunflower Oil-in-Water Emulsions with Antioxidant (α-Tocopherol) Either in Palmitin Colloidal Particles (the Emulsion of the Invention) or in the Core of the Oil Droplets

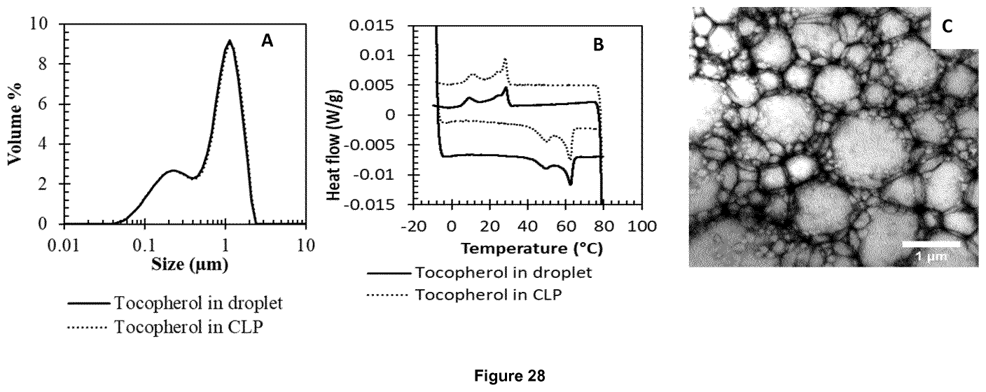

In this example, we characterized Pickering oil-in-water emulsions by measuring the droplet size distribution (A), their thermal properties of melting and crystallization (B), as well as their morphology (C) (FIGS. 28A, B and C).

### Example 20. Comparison of the Activity of α-Tocopherol-Loaded Colloidal Particles in the Sunflower Oil-in-Water Emulsions of Example 13 with an Emulsion Prepared Using a Non-Oxidizable Oil

To investigate whether α-tocopherol-loaded colloidal particles adsorbed at the droplet surface of Pickering emulsion prevents oxidation in the emulsion through a specific antioxidant action and not any other mechanism, the experimental set up of Example 3 was reproduced, except that the oil used in the emulsion consisted of medium chain triglycerides (MCTs) instead of stripped sunflower oil.

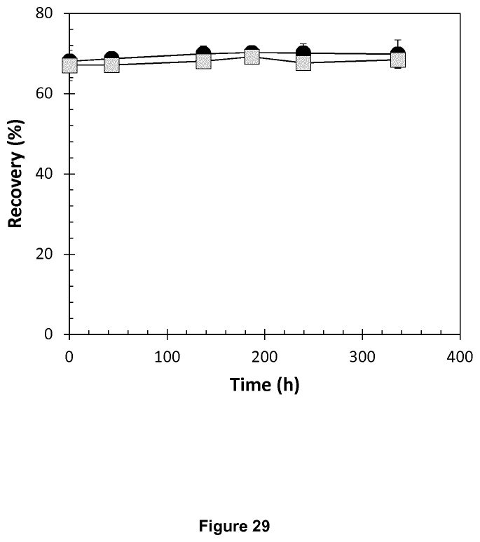

Unlike sunflower oil, MCTs are non-oxidizable and it can be seen in FIG. 29 that there was no significant consumption of α-tocopherol observed in either emulsion, even in the presence of ferrous iron (200 μM FeSO₄/EDTA at 25° C.). This suggests that α-tocopherol is specifically consumed by lipid oxidation products and not directly by cation metals. Consequently, α-tocopherol-loaded colloidal particles exert a protecting effect towards Pickering emulsions through a true antioxidative action.

### Example 21. Calorimetrical Characterization of a Conventional Sodium Caseinate-Stabilized Sunflower Oil-in-Water Emulsion Containing Added Palmitin Colloidal Particles in the Aqueous Phase (Solid Line), and a Colloidal Lipid Particles Dispersion (Dashed Line)

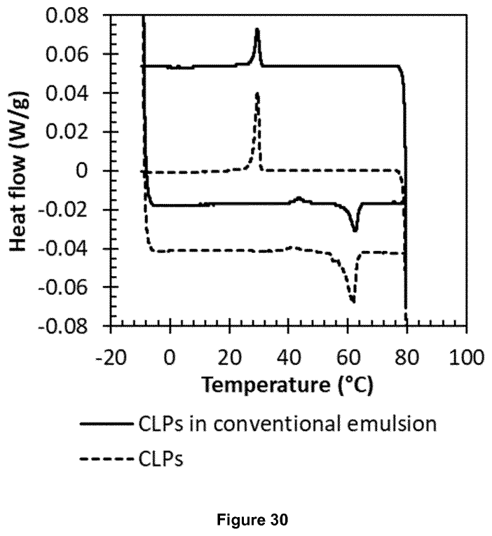

In this example, we characterized a conventional sodium caseinate-stabilized emulsion using differential scanning calorimetry (FIG. 30).

### Example 22. Comparison of Sunflower Oil-in-Water Emulsions where the Antioxidant (α-Tocopherol) is Either (i) in the Palmitin (80%) Colloidal Particles Containing 20% Tricaprylin (the Emulsion of the Invention Prepared as Detailed in the Materials and Methods Section) or (ii) within the Interior of the Oil Droplets

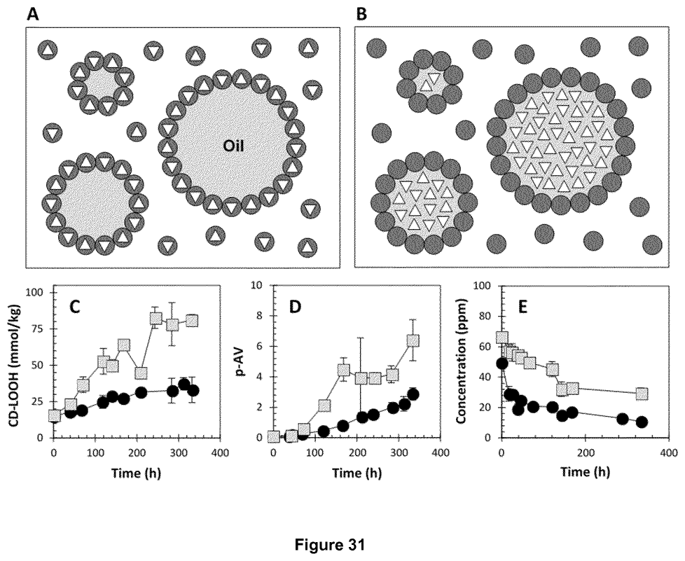

The exact same experimental design as Example 13 has been reproduced here but with colloidal particles containing 20% tricaprylin/80% tripalmitin instead of 100% tripalmitin (FIGS. 31A and B). The data presented in FIGS. 31C, D, and E clearly showed that the same type of advantage is obtainable with these colloidal particles, indicating that the antioxidant-enhancing effect is robust toward variation of the colloidal particle composition.

### Example 23. Comparison of Flaxseed Oil-in-Water Emulsions where the Antioxidant (α-Tocopherol) is Either (i) in the Palmitin Colloidal Particles (the Emulsion of the Invention Prepared as Detailed in the Materials and Methods Section) or (ii) within the Interior of the Oil Droplets

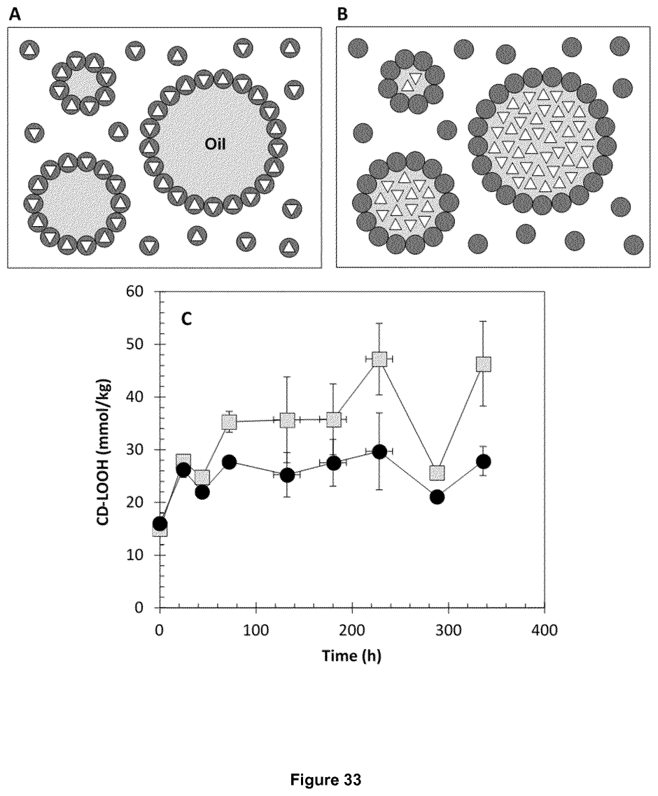

The exact same experimental design as Example 13 has been reproduced here but with flaxseed oil instead of sunflower oil (FIGS. 33A and B). The data presented in FIG. 33C clearly showed that the same type of advantage is obtainable with a different oil than sunflower oil, indicating that the antioxidant-enhancing effect is robust toward variation of the oil composition.

1. A composition comprising particles prepared from one or more biological materials that are capable of locating to or at an interface when combined with two or more immiscible liquids.

2. The composition according to claim 1, wherein the particles comprise biological materials selected from the group consisting of blue-green algae, the Rutaceae family, the Malvaceae family, the Rubiaceae family, the Amaranthaceae family, the Poaceae family, the Zingiberaceae family, the Ginkgoaceae, the Araliaceae family, the Theaceae family, the Asteraceae family, the Oleaceae family, the Moringaceae family, the Bromeliaceae family, the Brassicaceae family, the Rosaceae family, the Sapindaceae family, the Lamiacea family, and mixtures thereof and/or animal lipids and/or plant lipids selected from the group consisting of milk fat, palm oil, palm kernel oil, coconut oil, cuphea oil, cocoa butter, shea butter, tripalmitin, palm stearin, waxes, fractionated oils, hydrogenated oils, and mixtures thereof.

3. (canceled)

4. The composition according to claim 2, wherein the particles prepared from animal lipids and/or plant lipids are solid at room temperature.

5. (canceled)

6. The composition according to claim 1, wherein the particles have a diameter from about 0.1 μm to about 100 μm.

7. The composition according to claim 1, wherein the particles comprise an antioxidant.

8. The composition according to claim 7, wherein the anti-oxidant is in or from a plant or microalgal extract rich in antioxidants.

9. The composition according to claim 8, wherein the plant or microalgal extract rich in antioxidants is a rosemary, sage, or green tea extract, raw material or extraction cake, a Dunaliella salina extract or oleoresin, a spirulina extract or extraction cake, or a spinach extract, raw material or extraction cake.

10. (canceled)

11. The composition according to claim 7, wherein the antioxidant is selected from the group consisting of tocopherols, tocotrienols, plastochromanols, phenolic diterpenes, flavonoids, phenolic acids and esters, stilbenes, carotenoids, essential oils and mixtures thereof, and/or synthetic antioxidants selected from the group consisting of butylated hydroxyanisole (BHA), butylated hydroxytoluene (BHT), tert-butyl-hydroxyquinone (TBHQ), propyl gallate (PG), ascorbyl palmitate and mixtures thereof.

12. (canceled)

13. An emulsion comprising a composition comprising particles as defined in claim 1, the emulsion comprising an internal phase dispersed in a continuous external phase, wherein particles are located at the interface of the external and the internal phase and at least one of the internal or external phase comprises an oxidisable compound.

14. The emulsion according to claim 13, wherein the oxidisable material comprises a lipid.

15. The emulsion according to claim 13 or 14, wherein the lipid has at least one carbon-carbon double bond in the fatty acyl chain and is selected from the group consisting of palmitoleic acid, oleic acid, myristoleic acid, linoleic acid, arachidonic acid, linolenic acid, eicosapentaenoic acid, docosahexaenoic acid, sunflower, soybean, canola, rapeseed, flaxseed, olive, peanut, corn, cottonseed, palm, fish oils, and combinations thereof.

16. The emulsion according to claim 13, wherein the emulsion is an oil-in-water emulsion.

17. (canceled)

18. (canceled)

19. (canceled)

20. A method for reducing or preventing oxidation and/or enhancing the oxidative stability of an emulsion comprising either:
(ii) forming an emulsion comprising an internal phase dispersed in a continuous external phase and adding a composition comprising particles as defined in claim 1 to the emulsion; or
(ii) forming an emulsion comprising an internal phase dispersed in a continuous external phase and a composition comprising particles as defined in claim 1 by mixing two or more immiscible liquids and the particles under conditions suitable for forming an emulsion;
wherein at least one of the internal or external phase comprises an oxidisable material.

21. A method of prolonging the shelf-life of a beverage, a nutraceutical, a pharmaceutical or food product comprising an emulsion, wherein the method comprises either:
(iii) forming an emulsion comprising an internal phase dispersed in a continuous external phase and adding a composition comprising particles as defined in claim 1 to the emulsion; or
(iv) forming an emulsion comprising an internal phase dispersed in a continuous external phase and a composition comprising particles as defined in claim 1 by mixing two or more immiscible liquids and the particles under conditions suitable for forming an emulsion;
wherein at least one of the internal or external phase comprises an oxidisable material.

22. (canceled)

23. (canceled)

24. (canceled)

25. The method according to claim 20, wherein the oxidisable material comprises a lipid.

26. The method according to claim 25, wherein the lipid has at least one carbon-carbon double bond in the fatty acyl chain and is selected from the group consisting of palmitoleic acid, oleic acid, myristoleic acid, linoleic acid, arachidonic acid, linolenic acid, eicosapentaenoic acid, docosahexaenoic acid, sunflower, soybean, canola, olive, peanut, corn, cottonseed, palm, fish oils, and combinations thereof.

27. The method according to claim 20, wherein the internal phase comprises oil and the external phase comprises water.

28. (canceled)

29. (canceled)

30. (canceled)

31. The method according to claim 20, wherein the particles reduce, delay and/or prevent the formation of oxidation products, secondary oxidation products and/or non-volatile secondary oxidation products.

32. The method according to claim 20, wherein the emulsion is a nutraceutical composition, dietary or food product for humans or animals, nutritional supplement, fragrance or flavouring, pharmaceutical or veterinary composition, oenological or cosmetic formulation or the emulsion is part of a nutraceutical composition, dietary or food product for humans or animals, nutritional supplement, fragrance or flavouring, pharmaceutical or veterinary composition, oenological or cosmetic formulation.

33. A nutraceutical composition, a dietary or food product for human or animals, nutritional supplements, a fragrance or flavouring, a pharmaceutical or veterinary composition, an oenological or cosmetic formulation comprising a composition as defined in claim 1.

34. A method of utilizing the composition as defined in claim 1 comprising adding said composition to a nutraceutical composition, a dietary or food product for humans or animals, a nutritional supplement, a fragrance or flavouring, a pharmaceutical or veterinary composition, an oenological or cosmetic formulation.

35. A method for the preparation of an emulsion, wherein the method comprises:
mixing a composition as defined in claim 1 with either:
(c) two or more immiscible liquids; or
(d) a pre-prepared emulsion comprising an internal phase dispersed in a continuous external phase.

36. (canceled)

37. (canceled)

38. A kit for prolonging the shelf life of a beverage, a nutraceutical, a pharmaceutical or food product comprising an emulsion, wherein the emulsion comprises an internal phase dispersed in a continuous external phase, and at least one of the internal or external phase comprises an oxidisable material; the kit comprising particles as defined in claim 1.

39. The method according to claim 31, wherein the particles reduce, delay and/or prevent the formation of oxidation products including lipid hydroperoxides and conjugated diene hydroperoxides and/or secondary oxidation products including aldehyde, ketone, alcohol, and carboxylic acid volatile compounds and/or non-volatile secondary oxidation products including p-anisidine, epoxides, dimers and polymers.

40. A nutraceutical composition, a dietary or food product for human or animals, nutritional supplements, a fragrance or flavouring, a pharmaceutical or veterinary composition, an oenological or cosmetic formulation comprising a emulsion as defined in claim 13.
